(19) United States
(12) Patent Application Publication
(10) Pub. No.: US 2026/0355359 A1
(43) Pub. Date: Nov. 12, 2026

(54) Compositions, Systems and Methods for Radiotherapy

(71) Applicant: The Johns Hopkins University, Baltimore, MD (US)

(72) Inventors: Wilfred Ngwa, Baltimore, MD (US); Shahinur Acter, Baltimore, MD (US)

(21) Appl. No.: 19/210,774

(22) Filed: May 16, 2025

Related U.S. Application Data
(60) Provisional application No. 63/648,654, filed on May 16, 2024.
(60) Provisional application No. 63/651,732, filed on May 24, 2024.

(51) Int. Cl. A61K 49/18; A61K 31/704; A61N 5/10; A61P 35/00; C07K 16/28

(52) U.S. Cl. CPC A61K 49/1857; A61K 31/704; A61N 5/1001; A61N 5/1039; A61N 5/1077; A61P 35/00; C07K 16/2878

(57) ABSTRACT

Systems and methods for image-guided radiotherapy drug-delivery that include use of polydopamine materials. In preferred aspects, the present systems and methods include a therapeutic composition that comprises a polydopamine material associated with one or more therapeutic agents.

The present application claims priority from U.S. provisional application No. 63/648,654 filed May 16, 2024 and U.S. provisional application no. 63/651,732 filed May 24, 2024, both of which applications are incorporated by reference herein in their entirety.

### STATEMENT REGARDING FEDERALLY SPONSORED RESEARCH

This invention was made with government support under grant CA239042 awarded by the National Institutes of Health. The government has certain rights in the invention.

### BACKGROUND

Currently used traditional cancer therapies includes surgery, radiotherapy, chemotherapy, and immunotherapy, which have limitations in therapeutic efficacy and toxicities [1-4]. Therefore, cancer still remains a leading cause of death and morbidity in the world [2, 5].

It thus would be desirable to have new cancer therapies.

### SUMMARY

We now provide new compositions, systems and methods that can provide drug localization and activated release at a targeted treatment site, including a tumor site.

More particularly, we now provide systems and methods for image-guided radiotherapy drug-delivery that include use of polydopamine materials.

In particular aspects, the presents systems and methods include a therapeutic composition that comprises a polydopamine material.

In one aspect, a polydopamine material is utilized in the present compositions and methods that comprise one or more associated therapeutic agents, including anti-cancer agents. The therapeutic agent(s) may be bonded to the polydopamine material by covalent or non-covalent bonds. In particular aspects, the bonded therapeutic agents may dissociate from the polydopamine material at a treatment site, for example, the acidic environment of a tumor or cancer cells may result in cleavage or other degradation or reduction of covalent or non-covalent bonds that had linked the therapeutic agents to the polydopamine material, thereby between enabling effective administration of the therapeutic agents to the targeted treatment site.

In one aspect, systems are provided where a polydopamine material is used to provided image-guidance during radiotherapy. The polydopamine composition can be monitored or tracked e.g. by a suitable imaging tool such as photoacoustic imaging, magnetic resonance imaging and/or computed tomography (CT).

In particular aspects, a contrast agent such as a metal e.g. metal such as gadolinium may be incorporated with the polydopamine material to facilitate such imaging and image-guidance for radiation therapy.

The polydopamine composition can guide radiation therapy including external-beam radiotherapy (EBRT) with x-rays or ions (protons or carbon ions) to target a treatment site such as a tumor site and monitor treatment response. The radiation therapy can be either or both external (such as EBRT) or internal (such as brachytherapy). In aspects, external radiotherapy (such as EBRT) may be preferred.

In a further aspect, treatment methods comprising: a) administering to a subject in need thereof a polydopamine composition comprising one or more therapeutic agents; and thereafter b) administering radiation therapy preferably together with guidance of the administered polydopamine composition. In aspects, the radiation therapy causes the polydopamine composition to generate heat resulting from Cerenkov photons generated within the tumor by the administered radiation. The radiation therapy also can activate biodegradation of the polydopamine material to release the drug and by products associated with the polydopamine material. As discussed above, the released drugs and by products can further enhance damage to the tumor cells and in-situ vaccination, to significantly improve the outcomes of radiotherapy with minimal toxicity. In certain preferred aspects, the radiation therapy is external radiotherapy (such as EBRT). In certain preferred aspects, the radiation therapy is internal radiotherapy (such as brachytherapy).

In a further aspect, methods are provided comprising: a) administering a polydopamine composition comprising one or more therapeutic agents such as immune adjuvant drugs to a subject in need thereof such as a subject having cancer such as a tumor; b) acquiring images (e.g. magnetic resonance imaging) of the cancer and suitably surrounding tissues; and c) delivering radiation to the subject e.g. at the tumor site. In preferred aspects, the polydopamine composition can be activated by the radiation e.g. Cerenkov radiation generated by a high energy radiation beam. Preferably, radiation delivered to the subject can be aligned or adjusted as desired based on prior or current imaging of the subject e.g. imaging of the tumor site. More generally, treatment of the subject can be monitored and adjusted based on imaging of the subject and treatment site. In certain preferred aspects, the delivered radiation is external radiotherapy (such as EBRT). In certain preferred aspects, the delivered radiation is internal radiotherapy (such as brachytherapy).

In one aspect, present systems may comprise an external beam radiation source, an imaging device, and a controller.

The radiation source can be an EBRT source such as e.g. a linear accelerator or a cyclotron/synchrotron that produces and delivers beams of radiation to a treatment area such as a patient's tumor site. The radiation source for external radiation therapy suitably provides ionizing radiation to a defined target volume in a patient. External beam radiation therapy, also referred to as “teletherapy,” may suitably be administered by directing a linear accelerator (“linac”), or cobalt-60 (⁶⁰Co) teletherapy unit, to produce beams of ionizing radiation that irradiate the defined target volume in a patient. The radiation beam is a beam of radiation that may be delivered to the target region from several different directions, or beam paths.

The imaging device can be for example a photoacoustic imager, CT scanner, or MRI scanner, or device combining any of these modalities that acquires images of the tumor and the surrounding tissues before or during the radiation treatment.

The controller can be a computer or a processor that controls the operation of the radiation source, and the imaging device, based on pre-determined parameters or feedback signals. In distinction from prior approaches, the present systems do not need or require a light source to activate the polydopamine material. Rather, in preferred aspects, the present system can utilize radiation (e.g. Cerenkov radiation) generated during image-guided radiation therapy (IGRT) by the high-energy beams targeted to the tumor with the guidance of the polydopamine material.

Preferred polydopamine materials include polydopamine nanobowls (PNB). In preferred aspects, the PNB suitably can have e.g. a diameter or longest dimension of about 50 nm to about 600 nm, a wall thickness of about 10 nm to about 30 nm, and a cavity size of about 60 nm to about 140 nm. In preferred aspects, the PNB suitably can have a mesoporous structure with an average pore size of about 2 nm to about 10 nm and a surface area of about 100 m2 /g to over 500 m2 /g.

In certain preferred aspects, the polydopamine materials (e.g. PNB) can be loaded with one or more therapeutic agents including e.g. immune adjuvant drugs, such as STING agonists or anti-CD40, by incubating them in a drug solution for a certain period of time. The loading capacity and release kinetics of the drugs can be controlled by varying factors such as drug concentration, and time.

The drug-loaded polydopamine material can be administered to a subject having a tumor e.g. by injection or local intratumoral administration or inhalation. In aspects, the drug-loaded polydopamine material can accumulate in the tumor site by passive targeting through enhanced permeability and retention (EPR) effect or active targeting through surface modification with ligands that bind to specific receptors on the tumor cells. The drug-loaded polydopamine material can also be visualized by various imaging techniques, such as PAI, CT, or MRI, to guide the radiation therapy. The drug-loaded polydopamine material can enhance the contrast of the images due to their high density, and magnetic properties.

During the IGRT session, the drug-loaded polydopamine material can be activated by e.g. Cerenkov radiation from high energy EBRT beam to generate heat, enhance the release of the loaded drug agent(s) from the polydopamine material, and enhance damage to tumor cells. The Cerenkov radiation (UV light) is preferably generated within the tumor e.g. by EBRT guided by the polydopamine without causing significant damage to the normal cells.

In preferred aspects, the polydopamine component of the drug-loaded composition can absorb the UV light and convert it into heat through the polydopamine optical absorption and photothermal conversion efficiency.

The heat generated by the polydopamine material component can increase the local temperature of the tumor and induce hyperthermia, which can damage the cancer cells and increase damage to them. The heat and UV light can also trigger the release of the drugs from the PNB by breaking bonds (e.g. hydrogen bonds and/or covalent bonds) between the therapeutic agent(s) and the polydopamine material or increasing the diffusion rate of the therapeutic agent(s). The released therapeutic agent(s) can diffuse into the tumor cells and exert cytotoxic effects on local tumor cells of or prime in-situ vaccination causing immune-mediated tumor cell kill outside the radiation field.

In preferred aspects, radiation therapy (e.g. IGRT) in combination with drug-loaded polydopamine material can synergistically induce tumor cell death both within and outside the radiation field, while minimizing damage to healthy tissue due to the image-guidance, hence significantly improving therapeutic efficacy.

The ability to image the polydopamine material via various modalities allow for monitoring and quantifying the distribution of its components over time and correlation to treatment response. The systems and methods for image-guided radiotherapy drug-delivery with polydopamine materials can offer new opportunities for enhancing IGRT to treat both early and advanced disease states and monitor the treatment response.

In certain aspects, one or more therapeutic agents associated with polydopamine material as disclosed herein can dissociate from the polydopamine material (e.g. breaking of non-covalent (e.g. hydrogen bonds) or covalent bonds in an acidic (e.g. pH less than 7, 6.5, 6.0, 5.5, 4, 4.5 or 4) environment such as may be present in the in vivo environment of cancer cells or a tumor. In that way, the polydopamine material with associated therapeutic agent(s) may deposit the therapeutic agent(s) at a targeted treatment site of the cancer cells or tumor. See the examples which follow.

In a further aspect, new polydopamine materials are provided comprising one or more STING agonists, or anti-CD40 or other immunoadjuvant. In preferred aspects, the polydopamine material or composition further comprises a contrast agent or metal, for example gadolinium and/or gold, or a metal oxide. In preferred aspects, the polydopamine material or composition further comprises an imaging contrast agent, such as a magnetic resonance imaging (MRI) contrast agent such as gadolinium. In preferred aspects, the polydopamine material comprises polydopamine nanobowl particles.

In a yet further aspect, new polydopamine materials are provided comprising one or more of docetaxel, paclitaxel, doxorubicin, cisplatin, gemcitabine, enzalutamide, flutamide, nilutamide, bicalutamide, abiraterone (including abiraterone acetate), cyproterone (including cyproterone acetate), megestrol (including megestrol acetate), chlormadinone (including chlormadinone acetate), spironolactone, canrenone, drospirenone, dienogest, norgestimate, ketoconazole, or cimetidine, and combinations thereof. In preferred aspects, the polydopamine material or composition further comprises a contrast agent or metal, for example gadolinium and/or gold, or a metal oxide. In preferred aspects, the polydopamine material or composition further comprises an imaging contrast agent, such as a magnetic resonance imaging (MRI) contrast agent such as gadolinium. In preferred aspects, the polydopamine material comprises polydopamine nanobowl particles. In certain aspects, a polydopamine material is associated with one or more therapeutic agents that are not doxorubicin.

In a further aspect, new polydopamine materials are provided comprising one or more one or more checkpoint inhibitors (e.g. Pembrolizumab, Nivolumab, Atezolizumab, Ipilimumab, Tremelimumab, Avelumab, Durvalumab, Cemiplimab, and/or Dostarlimab). In preferred aspects, the polydopamine material or composition further comprises a contrast agent or metal, for example gadolinium and/or gold, or a metal oxide. In preferred aspects, the polydopamine material or composition further comprises an imaging contrast agent, such as a magnetic resonance imaging (MRI) contrast agent such as gadolinium. In preferred aspects, the polydopamine material comprises polydopamine nanobowl particles.

Definitions Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value or range. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

The term “administering,” as used herein, refers to any mode of transferring, delivering, introducing, or transporting an agent (such a polydopamine composition) to a patient, for example, to a subject in need of treatment for a disease or condition.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements-- or, as appropriate, equivalents thereof-- and that other elements can be included and still fall within the scope/definition of the defined item, composition, apparatus, method, process, system, etc.

“Diagnostic” or “diagnosed” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

As used herein, “nanoparticle” refers to a particle having a length in its longest dimension of between about 1 nm and about 999 nm. Nanoparticles for use in the invention are preferably very small nanoparticles having an average size in the range from about 2 nm to about 15 nm. In various embodiments, the nanoparticles can have an average (e.g., diameter, or other largest extent in one dimension) from about 1 nm to about 10 nm, from about 5 nm to about 20 nm, from about 10 nm to about 30 nm, from about 15 nm to about 30 nm, from about 20 nm to about 40 nm, from about 25 nm to about 50 nm, from about 30 nm to about 60 nm, from about 40 nm to about 80 nm, from about 50 nm to about 100 nm, or from about 10 nm to about 100 nm, or from about 20 nm to about 150 nm. In the composition, a fraction of the metallic nanoparticles may have a size as indicated above. For example, at least 50%, at least 60%, at least 70%, at least 80%, at least 90%, at least 95%, at least 96%, at least 97%, at least 98%, or at least 99% of the nanoparticles may have sizes as indicated above. The nanoparticles may be essentially bowl-shaped, or may have other shapes, such as spherical, rods, cylinders, cubes, ellipsoids, core-shell structures, or may be irregular in shape.

As referred to herein, polydopamine refers to a polymer material that can be produced by reaction of dopamine, for example a p olymer produced by the autoxidation of dopamine (monomer). Polydopamine materials can be synthesized as disclosed herein and as reported and are commercially available from Luna Nanotech and others.

As referred to herein, a therapeutic agent “associated” with polydopamine material indicates the therapeutic agent is present in admixture with polydopamine material including upon administration of the polydopamine/therapeutic agent admixture to a human patient or other subject, whereby the polydopamine and therapeutic agent admixture are administered together to the subject. In at least certain preferred aspects, an associated therapeutic agent may be bonded to the polydopamine material either through a covalent bond or a non-covalent bond such as hydrogen bonding. Preferably such covalent or non-covalent bonding is labile in an acidic environment such as that of cancer cells or tumor whereby the therapeutic agent may effectively dissociate from the polydopamine material within the location of the cancer cells or tumor. It will be understood however that a therapeutic agent is considered “associated” with polydopamine material herein if the therapeutic agent is physically present in admixture with the polydopamine material even if covalent or non-covalent (such as hydrogen bonding) bonding is considered to not exist or not exist to a significant extent (e.g. less than 5, 4, 3, 2 or 1 weight present of the therapeutic composition is bonded to the polydopamine material) between the therapeutic agent and the polydopamine material.t

An “effective amount” as used herein, means an amount which provides a therapeutic or prophylactic benefit.

The term “pharmaceutical composition” is meant any composition, which contains at least one therapeutically or biologically active agent and is suitable for administration to the patient. Any of these formulations can be prepared by well-known and accepted methods of the art. See, for example, Remington: The Science and Practice of Pharmacy, 20th edition, (ed. A. R. Gennaro), Mack Publishing Co., Easton, Pa., 2000.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

By “reduces” is meant a negative alteration of at least 10%, 25%, 50%, 75%, or 100%.

By “reference” is meant a standard or control condition.

The terms “sample,” “patient sample,” “biological sample,” and the like, encompass a variety of sample types obtained from a patient, individual, or subject and can be used in a diagnostic, prognostic and/or monitoring assay. The patient sample may be obtained from a healthy subject, a diseased patient, or a patient with lung cancer. In certain embodiments, a sample that is “provided” can be obtained by the person (or machine) conducting the assay, or it can have been obtained by another, and transferred to the person (or machine) carrying out the assay. Moreover, a sample obtained from a patient can be divided and only a portion may be used for diagnosis. Further, the sample, or a portion thereof, can be stored under conditions to maintain sample for later analysis. The definition specifically encompasses blood and other liquid samples of biological origin (including, but not limited to, peripheral blood, serum, plasma, cord blood, amniotic fluid, cerebrospinal fluid, urine, saliva, stool and synovial fluid), solid tissue samples such as a biopsy specimen or tissue cultures or cells derived therefrom and the progeny thereof. In certain embodiment, a sample comprises cerebrospinal fluid. In a specific embodiment, a sample comprises a blood sample. In another embodiment, a sample comprises a plasma sample. In yet another embodiment, a serum sample is used. The definition of “sample” also includes samples that have been manipulated in any way after their procurement, such as by centrifugation, filtration, precipitation, dialysis, chromatography, treatment with reagents, washed, or enriched for certain cell populations. The terms further encompass a clinical sample, and also include cells in culture, cell supernatants, tissue samples, organs, and the like. Samples may also comprise fresh-frozen and/or formalin-fixed, paraffin-embedded tissue blocks, such as blocks prepared from clinical or pathological biopsies, prepared for pathological analysis or study by immunohistochemistry.

As defined herein, a “therapeutically effective” amount of a compound or agent (i.e., an effective dosage) means an amount sufficient to produce a therapeutically (e.g., clinically) desirable result. The compositions can be administered from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compounds of the disclosure can include a single treatment or a series of treatments.

The terms “treating” and “treatment” as used herein refer to the administration of an agent or formulation to a clinically symptomatic individual afflicted with an adverse condition, disorder, or disease, so as to effect a reduction in severity and/or frequency of symptoms, eliminate the symptoms and/or their underlying cause, and/or facilitate improvement or remediation of damage.

Any compositions or methods provided herein can be combined with one or more of any of the other compositions and methods provided herein.

Other aspects are disclosed infra.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows a schematic diagram showing the functionalization of polydopamine bowl-shaped mesoporous nanoparticles (on top) and (on bottom) showing MRI contrast of the nanosystem in mice model.

FIG. 2 (includes FIG. 2A (includes 2Aa-g and 2B) Transmission electron microscope (TEM) image of PDA nanobowls before functionalization (FIG. 2Aa and FIG. 2Ab) with (FIG. 2Ab) being a high magnified view of PDA nanobowl (FIG. 2Aa). Figure (FIG. 2Ac) to (FIG. 2Ae) show PDA nanobowls coated with various concentration of gadolinium (40, 100, and 150 μg/mL respectively). Figures FIG. 2Af and FIG. 2Ag are PDA nanobowls designed with Au NPs with high magnified view of (FIG. 2Af) shown in (FIG. 2Ag). FIG. 2B: Showing the size range of the nanobowls, the data was generated by analysis hundreds of nanoparticles from TEM image and three different TEM images of each sample were analyzed.

FIG. 3 shows a UV-vis absorption spectra of polydopamine mesoporous nanobowls and gadolinium coated PDA mesoporous nanobowls.

FIG. 4 (includes FIGS. 4A and 4B) shows (A) Showing the distinctive DOX peak at around 590 nm confirms adsorption of DOX into the nanobowls, and (B) Standard curve of DOX.

FIG. 5 shows the MRI contrast of gadolinium coated PDA mesoporous nanobowls in agar gel phantom, image on the left: agar gel phantom only (1), PDA nanoparticles in phantom (2), and gadolinium coated PDA nanobowls (gadolinium (III) chloride (100 μg/ml)) (3), image on the right: agar gel phantom only (1), (2) and (3) gadolinium coated PDA nanobowls with 100 μg/ml and 150 μg/ml of gadolinium (III) chloride respectively.

FIG. 6 (includes FIGS. 6A and 6B) shows: (A) Showing the MTT assay of PDA mesoporous nanobowls without and with gadolinium coating in TC-1 cells, (con. of gadolinium 100 μg.ml), P value_ (Only cells vs. bowls_100 μg/mL: 0.8380, Only cells vs. bowls_500 μg/mL: 0.4842, Only cells vs. Gd_bowls_100 μg/mL: 0.3737, Gd_bowls_500 μg/mL: 0.4872, (B) MTT assay results showing biocompatibility of PDA mesoporous nanobowls on Human umbilical vein endothelial cells (HUVEC).

FIG. 7 shows confocal microscopy images of TC-1 cells, showing cellular up taken of polydopamine nanobowls (at around 200 nm sized) after 24 h of incubation., herein, ells were stained with DAPI (blue) and the red signal is coming from the fluorophore dye (rhodamine 6 g) attached to the PDA nanobowls.

FIG. 8 (includes FIGS. 8A and 8B) shows: FIG. 8A: MRI images of tumor showing PDA mesoporous nanobowls coated with 100 μg/mL of gadolinium) and tumor visualization over time and FIG. 8B: Quantitative analysis of MRI images showing nanoparticles and tumor volume and intensity over time.

FIG. 9 (includes FIGS. 9a-9e)shows transmission electron microscope (TEM) images of polydopamine mesoporous nanobowls coated with gadolinium (con. of gadolinium_100 μg/mL), before (a) and after (b) loading with Doxorubicin respectively. Figure (c) is showing the loading efficiency of dox in gadolinium coated PDA mesoporous nanobowls (con. of gadolinium_100 μg/mL) and figure (d) is the release of dox from gadolinium coated PDA mesoporous nanobowls (con. of gadolinium_100 μg/mL)in pH 5.6.

FIG. 10 shows the cumulative PTX release in various pH.

FIG. 11 shows the efficiency of PTX loaded PDA nanobowls in killing cancer cells (A549 cells) in compared with the free PTX (left: 48 hrs and right: 72 hrs).

FIG. 12 (includes FIGS. 12A and 12B). FIG. 12A, confocal microscopy images, (al) PDA mesoporous nanobowls only, (a2) Alexa fluoro700 anti human CD40, (a3) PDA mesoporous nanobowls loaded with Alexa fluoro700 anti human CD40 and (a4) Gadolinium (con. of gadolinium_100 μg/mL) coated PDA mesoporous nanobowls loaded with Alexa fluoro700 anti human CD40. FIG. 12B is fluorescence intensity profile of AntiCD40 (Alexa fluor 700) release from PDA mesoporous nanobowls in various pH environment.

FIG. 13 (includes FIGS. 13A-13F). In FIG. 13A: TEM images of Hela cells showing uptake of bowl-shaped polydopamine mesoporous nanoparticles. In FIG. 13B is the high magnified view of TEM image. FIGS. 13B and 13C show the impact of various treatments on Hela cells without and with NIR illumination (1 W cm⁻², 5 min) respectively. FIGS. 13E and 13F show the impact of various treatments on Hela cells without and with NIR illumination (1 W cm⁻², 5 min), respectively, herein green and red bars represent live and dead cells, respectively.

FIG. 14 shows confocal microscopy images of Hela cells after various treatments, herein, a2 to a4 only cells, b1 to b4 cells incubated for nanobowls for 24 h, c1 to c4 cells incubated with free DOX for 24 h, d1 to d4 cells incubated with DOX loaded nanobowls for 4 h, e1 to e4 cells incubated with nanobowls for 24 h and f1 to f4 cells were treated with NIR after 24 h of incubation with DOX loaded nanobowls. NIR illumination for 5 min, 1 W cm⁻².

Scale bars represent 20 μm. Confocal fluorescence images of Hela cells after various treatments, herein, a2 to a4 only cells, b1 to b4 cells incubated for nanobowls for 24 h, c1 to c4 cells incubated with free DOX for 24 h, d1 to d4 cells incubated with DOX loaded nanobowls for 4 h, e1 to e4 cells incubated with nanobowls for 24 h and f1 to f 4 cells were treated with NIR after 24 h of incubation with DOX loaded nanobowls. NIR illumination for 5 min, 1 W cm⁻². Scale bars represent 20 μm.

FIG. 15 (includes FIGS. 15A-15C) depicts a graph showing presentation of cell viability of A 549 cells after (a) 48 h and (b) 72 h of treatment with mPDA-PTX-nb and PTX (control) at varying concentrations and (c) cell viability of HUVEC after incubation with mPDA nano-bowls for 72 h. The data are expressed as mean ±standard deviation (SD) with a sample size of n=3, where * is p<0.05, **p<0.01, and ***p<0.001, indicating statistical significance between mPDA-PTX-nb and PTX.

### DETAILED DESCRIPTION

In aspects, we now provide Polydopamine Radioimmunotherapy with Image-guided Monitoring and Enhanced (drug) Release System PRIMERS, functionalized polydopamine (PDA) including polydopamine bowl-shaped mesoporous nanoparticles (PDA nanobowls) that can serve as fiducial markers including for cancer therapy. By leveraging the advantages of excellent surface functionalization capability of PDA, anisotropic nanostructure, polydopamine has been successfully functionalized with imaging agent gold and gadolinium (MRI imaging agent), which shows strong MRI contrast signal both in-vitro in phantom and in-vivo in animals. As set forth in the examples which follow, the present polydopamine materials with anti-cancer drug loading are demonstrated to release the anti-cancer drugs effectively at a target treatment site. Thus, in one aspect, gadolinium coated PDA nanobowls demonstrate capacity for loading immunotherapy drugs (e.g. Anti-CD40) with activated release in acidic pH levels characteristic of the tumor environment with enhanced release following administration of radiation therapy. The results demonstrate combining radiotherapy with activated image-guided drug delivery, which can provide effective cancer treatment.

Multifunctional nanoparticles are increasingly desired to obtained enhance therapeutic efficacy and minimize the side effects in conventional cancer treatment [6-10]. In particular, combined immunotherapy and radiation therapy along with chemotherapy may potentiate significantly enhanced treatment efficacy, as well as image-guidance capability, which is valuable for guiding treatment and monitoring drug delivery [11-15].

Fiducial markers can be used to provide image-guidance during radiotherapy providing capability to accurately track the location and position of the tumors, especially for tumors that may move during the treatment [16, 17]. In a number of studies, fiducial markers demonstrate capability in improving the precision of radiation therapy, thus minimizing damage or toxicity to surrounding healthy tissues, which eventually improves the therapeutic efficacy [18, 19]. Since 1990s, fiducial markers have been playing an essential role in image-guided radiotherapy for cancer treatment [20]. Basically, fiducial markers are surgically implanted inside the location of interest prior to radiotherapy [19, 21]. To ensure positioning accuracy, fiducial markers are engineered to be implanted in the location of interest and are currently in solid or liquid forms [18, 20] [22]. Organic dyes attached to particles is one of commonly used fiducial markers that showed contrast in electron microscopy and CLEM [23].

However, this type of organic dye-based fiducial markers comes with the limitation of photobleaching as they are not well stable and electron beam exposure [22, 24]. More recently, increasing number of studies have investigated, injectable fiducial markers based on nanocarriers [16, 25, 26].

Nanoparticles based fiducial markers are designed to be bio-compatible and intrinsically non immunogenic without considering the ability to provide additional functions beyond image-guidance such as for image-guided drug delivery of immunotherapy, or chemotherapy [26-28].

In the development of multifunctional nanoparticles for combination cancer therapy, polydopamine (PDA) has gained due to the flexibility in designing desired morphology and size along with suitable surface functionalization properties [2, 29-31]. Moreover, PDA is known for its excellent biocompatibility, biodegradability, and colloidal stability in the biological system [32]. Among various size and shapes of PDA nanoparticles, bowl-shaped PDA mesoporous nanoparticles (PDA nanobowls) may be preferred including as multifunctional nanomedicine for cancer therapy [33]. PDA nanobowls have shown efficacy in combination cancer treatment with chemo and photothermal therapy [30]. Moreover, higher cellular up taken efficiency due to their bowl-shape and capability of loading sufficient amounts of drugs and molecules into the mesopores and cavity makes this anisotropic shaped nanoparticle unique [30, 33].

Polydopamine (PDA) contains active functionalities such as catechol and amino groups whereby the polydopamine can chelate metal ions or bond (e.g. both covalently and non-covalently such as hydrogen-bonding).

As discussed, a metal may be incorporated the polydopamine material to facilitate imaging. For instance, in a preferred aspect, to provide effective magnetic resonance imaging (MRI) contrast of polydopamine material, gadolinium may be incorporated with the polydopamine material.

As discussed, the polydopamine material also preferably contains one or more associated therapeutic agents. A wide variety of therapeutic agents may be associated with a poydopamine material including for example docetaxel, paclitaxel, doxorubicin, cisplatin, gemcitabine, a hydrophobic drug, an anti-androgen compound, a small molecule signaling pathway inhibitor, and combinations thereof. Exemplary anti-androgen compounds include enzalutamide, flutamide, nilutamide, bicalutamide, abiraterone acetate, cyproterone acetate, megestrol acetate, chlormadinone acetate, spironolactone, canrenone, drospirenone, dienogest, norgestimate, ketoconazole, cimetidine, and combinations thereof. Exemplary small molecule signaling pathway inhibitor is selected from the group consisting of a PI3K inhibitor, a PARP inhibitor, a PI3K/AKT/mTOR pathway inhibitor, and combinations thereof.

In certain embodiments, a polydopamine material may be conjugated to a tumor-targeting moiety (also referred to as a “targeting moiety”). The tumor-targeting moiety can be for example a tumor-targeting ligand, peptide, protein, aptamer, oligonucleotide, antibody, cell adhesion molecule, or small molecule. A “small molecule” as used herein refers to an organic molecule, such as a drug or metabolite, which is less than 1000 in molecular weight, and which binds to a target, such as a protein or nucleic acid, within or on the surface of a tumor cell. For example, a tumor-targeting ligand can be folic acid, an antibody, such as a single-chain variable fragment antibody, a ligand for an epidermal growth factor receptor, transferrin, an arginylglycylaspartic acid (RGD) peptide, riboflavin, a prostate specific membrane antigen (PSMA) aptamer, or a galactose derivative. As nanoparticles conjugated to a targeting moiety are released from the degrading matrix material, they can bind to tumor cells, and are therefore useful for cancer diagnostic or therapeutic purposes.

Immunoadjuvants or other molecules, such as targeting moieties, can be covalently or non-covalently conjugated to a polydopamine material by any of a variety of known methods and chemistries. For example, polydopamine materials contain various functional nanoparticles can be pre-functionalized with heterobifunctional polyethyleneglycols (PEGs) using a simple ligand exchange process. (Kumar et al. Translational Cancer Research. 2013, 2(4), 228-239).

Polydopamine materials as disclosed herein can be readily prepared. Exemplary protocols for preparing preferred polydopamine materials are set forth in the examples which follow. Polydopamine materials also are commercially available such as from Luna Nanotech. In one synthesis protocol, polydopamine (PDA) nanobowls can be prepared as an emulsion-induced interfacial anisotropic assembly [34]. Briefly, dopamine hydrochloride (DA) and a polymer such as Pluronic F127 (block copolymer) are admixed in a suitable solvent such as water: ethanol followed by addition 2,4-trimethylbenzene (TMB). Base is added followed by isolation of the reaction product such as by centrifuging.

The prepared polydopamine material have a metal or contrast agent admixed therewith for example by admixing the polydopamine material and a metal halide such as gold trichloride or gadolinium chloride (III). In one protocol, the gadolinium chloride (III) can be added during the polymerization to provide the polydopamine material. Suitable solvents include water and mixed solvent systems such as water: ethanol (e.g. 1:1 water: ethanol).

The prepared polydopamine material can be associated (e.g. covalent or non-colvelnt bonding) with one or more therapeutic by admixing of the prpapred polydopamine material and a drug agent in an aqueous or organic solvent system, preferably with agitation (stirring) and optionally with heating (e.g. to 30 °C, 35 °C, 40 °C or more) and optionally with adjusting pH to acidic or basic to facilitate bond formation between the polydopamine material the one or more therapeutic agents. A polydopamine material suitably may be associated with two or more distinct therapeutic agents, or only a single therapeutic agent. Exemplary preferred protocols for associating a therapeutic agent with polydopamine material are set forth in the examples which follow.

### Methods of Treatment

As discussed, a polydopamine material as disclosed herein associated with one or more therapeutic agents can be administered to treat a variety of diseases and disorders including cancer.

In aspects, the subject may be identified as suffering from pancreatic cancer, lung cancer, prostate cancer, breast cancer, colorectal cancer, liver cancer, cervical cancer and/or other gynecologic cancers. In aspects, the cancer may be metastatic cancer.

In aspects, the one or more agents associated with a polydopamine material can include an anti-cancer drug, such as for example, docetaxel, paclitaxel, doxorubicin, cisplatin, or gemcitabine, an anti-androgen compound, a small molecule signaling pathway inhibitor, or a combination thereof. Anti-androgen compounds can include, for example, enzalutamide, flutamide, nilutamide, bicalutamide, abiraterone acetate, cyproterone acetate, megestrol acetate, chlormadinone acetate, spironolactone, canrenone, drospirenone, dienogest, norgestimate, ketoconazole, or cimetidine. Small molecule signaling pathway inhibitors can include, for example, P13K inhibitors, PARP inhibitors, or P13K/AKT/mTOR pathway inhibitors. The therapeutic agent can also be released from nanoparticles, liposomes, polymersomes, or other carriers, providing a dual release mechanism.

In aspects, the one or more agents associated with a polydopamine material can include an immune adjuvant drugs, such as STING agonists (e.g. ADU-100, E7766 and GSK3745417) and/or anti-CD40, and/or checkpoint inhibitors (e.g. Pembrolizumab, Nivolumab, Atezolizumab, Ipilimumab, Tremelimumab, Avelumab, Durvalumab, Cemiplimab and. /orDostarlimab).

In one aspect, in methods for treating cancer, a composition comprising a polydopamine material as disclosed herein can be administered to a subject in need thereof e.g. by injecting or implanting the polydopamine a composition of the invention, or a device including the polydopamine composition, into a tumor or into a region of an organ or tissue that contains tumor cells in a patient in need thereof. In some embodiments, a brachytherapy spacer, radiotherapy fiducial marker, balloon applicator, brachytherapy applicator, transponder, or gel including or formed of the polydopamine composition is injected or implanted which can provide for internal radiotherapy. In brachytherapy (internal radiation therapy), x-ray-emitting radioisotopes (in the form of metallic seeds) may be implanted into tissue to be treated. In certain aspects, the method further includes performing radiation therapy on the patient.

In certain aspects, brachytherapy is performed, and an implanted brachytherapy spacer includes a polydopamine composition as disclosed herein.

As discussed, in preferred aspects, external beam therapy is performed and an implanted fiducial marker includes the polydopamine composition. In some aspects, the polydopamine composition includes gold-containing polydopamine nanoparticles, and the radiation therapy produces electrons by the photoelectric effect. The radiation used to treat the patient may be adjusted if necessary to include an energy level suitable for producing free electrons via the photoelectric effect from gold or another metal that may be included in the polydopamine composition. In additional aspects, a polydopamine composition may include titanium, and the radiation therapy produces Cherenkov radiation. Such radiation is known to produce reactive radicals that are toxic to tumor cells.

In aspects, light such as near infrared light and/or thermal (e.g. photothermal treatment) treatment is not substantially used to activate a composition comprising one or more associated drug agents for treatment of cancer cells, a tumor or other therapy. Instead, in preferred aspects, a radiation source such as external beam therapy or brachytherapy particularly that provides external beam therapy is used to active a drug-loaded polydopamine composition for treatment of cancer cells a tumor or other disease or disorder, including to activate such as by dissociation of the therapeutic agent(s) (e.g. breaking of covalent bonds or of hydrogen bonds or other non-covalent bonds) from the polydopamine material.

### Pharmaceutical Compositions

Such compositions typically include the agent and a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

In one embodiment, the polydopamine material suitably associated with one or more therapeutic agents are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid.

Toxicity and therapeutic efficacy of polydopamine material suitably associated with one or more therapeutic agents can be determined by standard pharmaceutical procedures in cell cultures or experimental animals, e.g., for determining the LD₅₀ (the dose lethal to 50% of the population) and the ED₅₀ (the dose therapeutically effective in 50% of the population).

The data obtained from cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For a compound used in a method of the disclosure, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography. Preferred dosages of a polydopamine material associated with one or more therapeutic agents may match or correspond to the same or similar (within 10, 20, 30 or 40 weight percent) of recommended dosages of the one or more therapeutic agents in the absence of the polydopamine material.

The pharmaceutical compositions can be included in a kit, container, pack, or dispenser together with instructions for administration.

The following examples are put forth so as to provide those of ordinary skill in the art with a complete disclosure and description of how to make and use the assay, screening, and therapeutic methods of the disclosure, and are not intended to limit the scope of what the inventors regard as their disclosure.

### EXAMPLES

### Materials and Methods

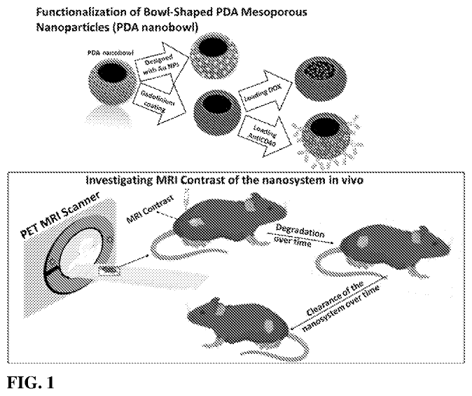

The Polydopamine Radioimmunotherapy with Image-guided Monitoring and Enhanced (drug) Release System (PRIMERS) are developed by functionalizing PDA nanobowls via coating with metal (gold or gadolinium) followed by loading of anticancer drugs whose release can be activated by external stimulus. A Schematic diagram is shown in FIG. 1.

### 2.1. Formation and Characterization of PDA Nanobowls

PDA nanobowls were designed following an established method known as an emulsion-induced interfacial anisotropic assembly [34]. Briefly, 1.5% (w/v) dopamine hydrochloride (DA) and 1.0% (w/v) Pluronic F127 (block copolymer), were dissolved in water: ethanol (1:1) mixture (total volume 10 mL) followed by addition (dropwise) of 2.0% (v/v) ,2,4-trimethylbenzene (TMB) under stirring. Ultra-sonication was used for 2 min to form the emulsion system. Alkaline environment was created by adding ammonia solution (3.75% (v/v), 28%) dropwise into the emulsion system. After 2 hours synthesized particles were centrifuged with water and ethanol mixture three to four times. The final product was freeze-dried for characterization and future use. Transmission electron microscopy (TEM (Hitachi 7600 TEM operating at 80 kV with an AMT XR80 CCD (8 megapixel)) were used to examine the size and morphology of the particles. Fluorescence, absorption and emission spectra of samples were recorded using a spectraMax Fluorescence spectrophotometer in 1 cm quartz cuvettes.′

### 2.2. Designing of PDA Nanobowls with Au Nanoparticles

2 mg/mL concentrated PDA nanobowls were dispersed in a 20 mL gold trichloride (5 mg/mL) aqueous solution. This mixture was stirred slowly for 30 min at 90 °C. Afterwards, the reaction was stopped by centrifugation three to 4 times using ultrapure water. The Au designed PDA nanbowls were collected and freeze dried for characterization and future use. TEM imaging analysis demonstrate successfully deposition of Au NPs on the surface of PDA nanobowls.

### 2.3. Coating With Gadolinium

In order to achieve magnetic resonance imaging (MRI) contrast of PDA nanobowls, a thin layer of gadolinium coating was created by adding gadolinium chloride (III) (from Sigma Aldrich) into the solvent (water: ethanol) of the nanoparticle's synthesis process. Herein, various concentrations of gadolinium chloride solution (40, 100 and 150 μg/mL) were used, which produced PDA bowls with various thickness of gadolinium on the surface. After the polymerization time (2 h), the PDA nanobowls were washed four to five times to remove free gadolinium. All supernatants were collected and for further analysis.

### 2.4. Loading Doxorubicin (DOX) into Gadolinium Coated PDA Nanobowls

To evaluate the drug delivery properties of gadolinium coated PDA nanobowls, the anti-cancer drug doxorubicin (DOX) was loaded into the nanobowls building on previous study [30]. For loading DOX, a 1 mg/mL nanobowls (total volume 5 mL) suspension was prepared, and various quantities of DOX (0.5, 1.0 1.5, 2.0, 2.5, and 3 mg) were dispersed in the nanobowls suspension and this mixture was stirred for 24 h at room temperature to allow binding to the nanobowls. Afterward, ultrapure water was used to wash the DOX loaded gadolinium coated PDA nanobowls by centrifugation three to four times to remove free DOX from the nanosystem. This process ended by freeze-drying the resulting dox-loaded PRIMER for future use. To estimate the drug loading capacity of the PRIMER, all supernatants were collected.

Herein, the loading efficiency of DOX was determined by fluorescence spectrophotometry (microplate reader spectramax) at an excitation wavelength of 480 nm and an emission wavelength of 590 nm by comparison to a standard curve (FIG. 4). DOX loading efficiency into the nanosystem was calculated by subtracting the mass of the DOX in the supernatant from the total mass of the DOX in the initial solution divided by the total amount of DOX and expressing it as a percentage.

### 2.5. Loading Anti CD40 in Gadolinium Coated PDA Nanobowl

To investigate the AntiCD40 loading efficiency in the PRIMERS, Alexa fluoro700 anti human CD40 (Vol. 5 μL, Con.200 μg/ml) was added in the particles suspension (50 mg/mL nanobowls (total volume 20 mL). The suspension was prepared in 25 mM MES buffer pH6)[35]. The loading experiment was conducted at 4 °C by slowing shaking for overnight. Afterward, antiCD40 load PRIMERS were washed several times (four to five times) by centrifugation at 13000 rpm to remove the free antiCD 40. All experimental procedures here were conducted at 4 °C in a sterile environment. All supernatants were collected for further analysis. Fluorescence microscopy (Zeiss LSM880-Airyscan FAST Super-Resolution) was used to characterized the PRIMERS.

### 2.6. MR Imaging

To assess ability to provide image contrast for treatment guidance and drug delivery monitoring, MRI imaging of the PRIMERS was conducted both vitro and vivo using 7T Bruker PET-MR Scanner. For in vitro imaging the sample was injected in agar gel phantom. The same concentration and volume of PRIMER components was injected in each phantom. For in vivo experiments, MRI imaging was investigates in C57BL6 mice bearing cervical subcutaneous tumors. MRI images were collected over different time points and the imaging was done over several weeks. All experiments were performed under anesthesia.

### 2.7. In Vitro Cytotoxicity Assay

Cell viability of the designed PRIMERS was determined using MTT cell viability assay. TC-1 cells were first seeded at a density of 2×10⁴ cells mL⁻¹ in a 96-well plate with a volume of 50 μL in each well and left to adhere for 24 h at 37 °C and 5% CO₂. The assay was conducted in triplicates with various concentrations with nanobowls that are noncoated and gadolinium coated ones (100 and 500 μg mL⁻¹) for 24 h at 37 °C under 5% CO₂. After the desired incubation time, the absorbance at 550 nm was recorded using a SpectraMax microplate reader. The absorbance values of the control (untreated cells) were set at 100%, and cell proliferation was expressed as a percentage of control. This study was conducted to investigate the cytotoxicity of the PRIMER components without the drugs loaded into them.

### 2.8. TC1 Cell Culture and Tumor Growth in C57BL6 Mice

A cervical cancer cell line, TC1 (ATCC® CRL-2785, Manassas, VA, USA), was used to grow subcutaneous tumors in C57BL6 female mice following previously published protocol [36]. The TC1 cells were cultured in RPMI 1640 medium supplemented with 2 Mm L-glutamine adjusted to contain 1.5 g/L sodium bicarbonate, 4.5 g/L glucose, 10 mM HEPES, 1.0 mM Sodium Pyruvate, 0.1 mM non-essential Amino Acids, and 10% fetal bovine serum in a humidified incubator at 37 °C under 5% CO₂. C 57BL/6 strain female mice were acquired from Charles River at 6-8 weeks old. They were inoculated subcutaneously with 3×10⁵ cells mixed with matrigel in the lateral flanks of mice and allowed to grow to a palpable tumor size of approximately 3 mm in diameter within ten days before the start of treatment. The formula: V=0.5×length×(width²) used determined mice tumor volume. To measure the longitude protrusion of the tumor a digital caliper was used, designated as the tumor length and the latitude projection as the tumor width. All the animal experiments were conducted as per the guidelines and regulations of the Johns Hopkins University Animal Care and Use Committee (ACUC) set under protocol #MO21M281. Mice maintenance in the Johns Hopkins University animal facility was according to the Institutional Animal Care and Use Committee approved guidelines.

### 3. Results and Discussions

### 3.1. Preparation and Characterization of PRIMERS Precursor Polydopamine Bowl-Shaped Mesoporous Nanoparticles (PDA Nanobowls)

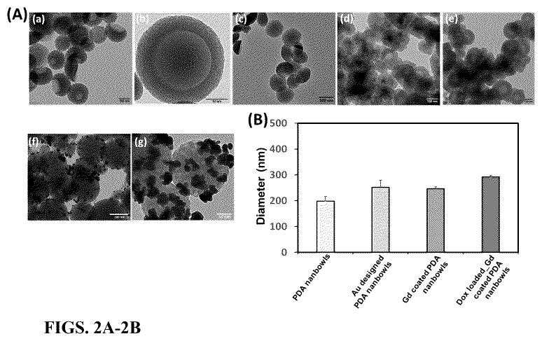

PRIMERS are developed from PDA nanobowls which serve as a precursur for functionalization and drug loading. In our study, an emulsion induced interfacial anisotropic assembly method was used to synthesize PDA nanobowls. Synthesis process of this anisotropic nanobowls is mainly based on the simultaneous formation of Pluronic® F-127/TMB/PDA composite micelles and nucleation of PDA and subsequent anisotropic growth of PDA on the surface of the emulsion droplet templates [34]. Previous studies demonstrate that by simply adjusting the concentration of pore swelling agent TMB, surfactant F127, monomer (dopamine), and aqueous ammonia solution the size and morphology of the PDA nanobowls can be controlled [29]. In FIG. 2A (a), monodispersity of the nanobowls is observed in TEM image, where the diameter of the nanobowls were found at around 200 nm and a cavity with an approximate width of 80 nm (FIG. 2a). Higher magnification revealed the pore size of the nanobowls, which is estimated to be around 7 nm (FIG. 2A (b)). Additionally, in all the TEM images as represented in FIG. 2A a and b, clear view of the mesochannels is achieved radially from the center to the surface of the bowls and the center-to-center distance between adjacent mesochannels is estimated to be ≈21 nm by higher magnification TEM image. In previous studies we have demonstrated the size distribution and zeta potential of PDA nanobowls for this formation process using dynamic light scattering (DLS) and phase analysis light scattering (PALS) respectively, where negative zeta potentials of the nanobowls with moderate magnitudes in both water and cell culture medium, suggesting colloidal stability [30, 33]. Previously, chemical characterization of the PDA nanobowls was also accomplished by Fourier transform infrared spectroscopy (FTIR) [33].

### Functionalization of PDA Nanobowls

PDA Nanobowls, Designed with Au Nanoparticles (NPs):

Due to the active surface property of having functional groups such as catechol groups, aromatic groups, amines and imines, PDA welcomes various metallic materials to be coated or deposited on its surface. Herein, taking the advantages of capping and reducing agents of Au, it has been successfully deposited on the surface of the PDA core shell nanoparticles [37]. TEM images in FIG. 2 f and g are showing the deposition of Au NPs on the PDA nanobowls, with high magnified view demonstrating the size of the Au functionalized PDA nanobowls, which slightly increases as observed in FIG. 2B. Later on, gadolinium was also used to coat on the surface of PDA nanobowls for comparison. Given the potential of gadolinium coated PDA nanbowls to provide both computed tomographic (CT) and magnetic resonance (MR) imaging contrast, these were further investigated in this study [38, 39].

### Gadolinium Coating on the PDA Mesoporous Nanobowls:

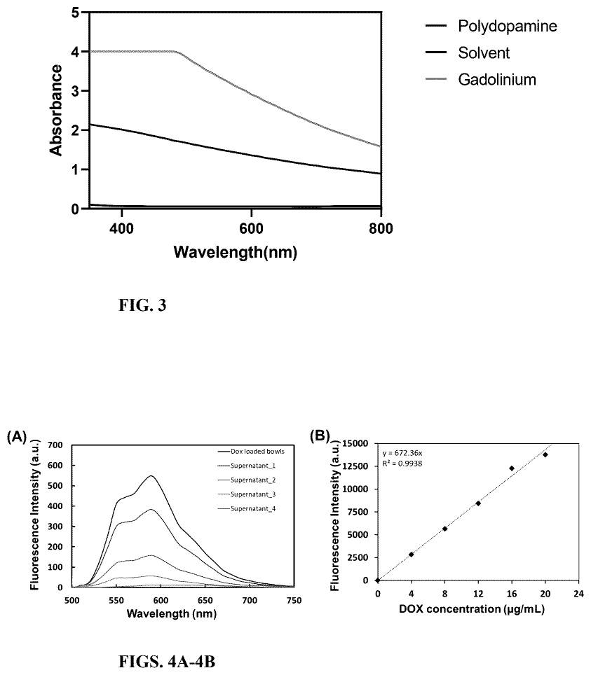

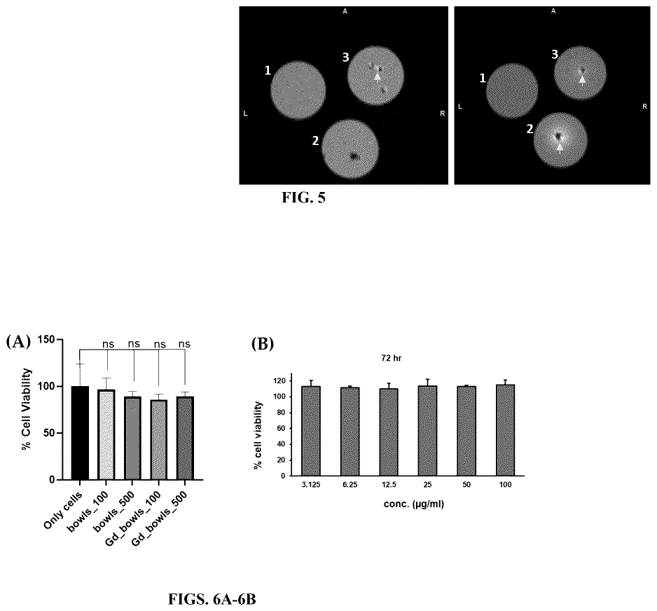

As mentioned earlier, PDA surface is designed with catechol-based functional groups, which has strong capability in forming coordination bonding with metal ions [2]. Taking the advantages of catechol-based functional groups on PDA, surface-controlled coating with gadolinium was achieved on the PDA mesoporous nanobowls in situ. In a series of experiments, various concentration of gadolinium chloride was used to coat on the surface of PDA nanobowls. To confirm the coating of gadolinium, TEM imaging analysis was conducted. TEM images in FIG. 2A (c to e) indicate that, with increased concentration of gadolinium chloride the thickness of the gadolinium on the PDA surface increased, where after thin coating on PDA surface the mesopores are clearly observed with slight increase in the size of the nanobowls (FIG. 2A (c). Moreover, the mesopores were filled with gadolinium (FIG. 2A (d) and (e). Uv-vis absorption spectra showing the presence of the gadolinium in the PDA nanobowls suspension is shown in FIG. 3. In a number of studies, it has been demonstrated that gadolinium-based nanoparticles can provide imaging contrast during magnetic resonance imaging (MRI) and boost the efficacy of radiation therapy. For MRI gadolinium is commonly used because of its seven unpaired electrons which benefited it with one of the most paramagnetic stable metal ions. Gadolinium molecules shorten the spin-lattice relaxation time (T1) of voxels in which they are present resulting in a brighter signal on T1 weighted images. The magnetic properties of gadolinium or gadolinium-based contrast agent in combination with magnetic resonance imaging (MRI) allow blood vessels, organs and other non-body tissues to be seen clearly during MR imaging, therefore, this rare earth metal is commonly applied in diagnosis and image-guidance therapy [38, 40]. To investigate the potential of gadolinium coated PDA mesoporous nanobowls as an MRI contrast agent for PRIMERS, a 7-tesla (T) MRI scanner was used. As demonstrated compared to the control 1 (only phantom) and control 2 (phantom injected with PDA nanobowls without gadolinium coating) the samples (PDA nanobowls with gadolinium coating) provide enhanced positive contrast during MRI scanning (FIG. 5).

From results, in phantom image the images evidently become brighter as the gadolinium concentrations increased, consistent with expectation that increased concentration of gadolinium in the nanosystem accelerates the recovery of net magnetization.

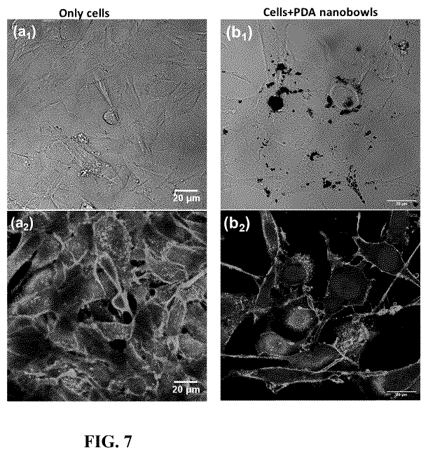

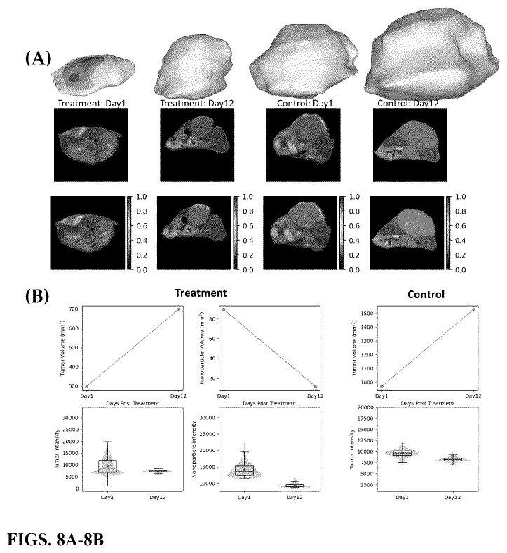

Before investigating the potential of the gadolinium coated PDA nanobowls as fiducial markers in vivo, its toxicity needed to be evaluated in vitro. The cell viability of the nanosystem has been investigated in TC-1 cells (FIG. 6A) and Human umbilical vein endothelial cells (HUVEC) (FIG. 6B) by using MTT assay kit, which showed no obvious cytotoxicity with the incubation of gadolinium coated PDA nanobowls. This was observed even with the concentration of the nanoparticles reached 500 μg/mL, with over 80% of the cells viable after 24 h of incubation time (FIG. 6A). Obtained results suggests biocompatibility of PDA nanobowls (FIG. 6B). Additionally, cellular internalization study of PDA nanobowls in TC-1 cells was conducted, and confocal microscopy images in FIG. 7 show a significant number of PDA nanobowls were taken up by the TC-1 cells in 24 h. In this study in vivo experiments were conducted in mice model by injecting the gadolinium coated PDA nanobowls intratumorally.

Positive MRI signal contrast was also observed in T1 weighted images of the tumor after the intra tumoral injection of the gadolinium coated PDA nanobowls. As observed in FIG. 8, sustained image contrast is observed up to at least 7 days post injection of the nanosystem, however, significant fading of the MRI signal observed from Day 12 until the tumor reached to the end point (FIG. 8). These results confirm the potential of the gadolinium coated PDA nanobowls as fiducial markers in vivo, especially for image-guided hypofractionated radiotherapy or radioimmunotherapy as shown in previous work using one radiotherapy treatment fraction. The system can be further optimized to customize to treatment schedules. The results also suggest the clearance of the nanoparticles from the biological system after a certain period of time due to the biodegradation of the nanoparticles. These results highlight the potential of the gadolinium coated PDA nanobowls as biodegradable fiducial markers in vivo.

### PRIMERS: Loading Anti-Cancer Drug in Gadolinium Coated PDA Nanobowl and Drug Release Profile In Vitro

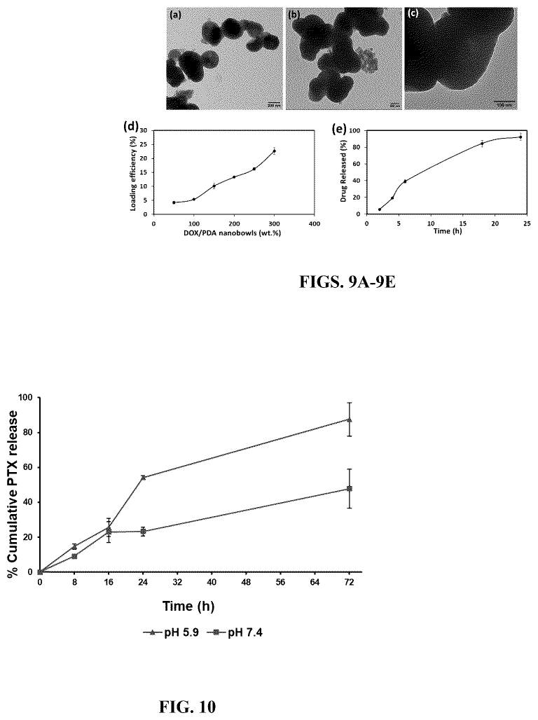

PDA nanobowls are rich with active functional group and aromatic rings, which allow them loading various types of anti-cancer drugs including doxorubicin (DOX). Herein, using aromatic rings of PDA, DOX molecules attached to the gadolinium coated PDA nanobowls by π-π stacking interactions. TEM images in FIG. 9 a and b showing the thickness gadolinium coated PDA nanobowls increased significantly after loading with DOX, high magnified view in FIG. 9c suggesting the deposition of DOX on the surface of the nanobowls and inside the cavity and mesochannels are also filled with DOX molecules. The distinctive peak at around 590 nm confirms the adsorption of DOX into the nanosystem. These samples were examined after loading the DOX and washing out the unbound DOX from the suspension (FIG. 4). As observed, in comparison to the gadolinium coated PDA nanobowls, the dox concentration in the supernatant decreases significantly in every wash and after the fourth wash DOX has been removed completely from the nanosystem (FIG. 4). Higher loading efficiency was observed from the noncoated PDA nanobowls [30] compared to the gadolinium coated one (FIG. 9d), it is assumed that the space of the nanobowls is partially preoccupied due to the coating of gadolinium. However, the loading efficiency is still higher compared to nonporous PDA nanobowls, which indicates a higher capacity of loading drugs in to the PDA nanobowls, confirming strong contribution of electrostatic attractions addition to the to π-π stacking in to the drug loading process due to their negative surface chare. It is assumed that considerably larger available space such as mesopores and cavity allow greater drug adsorption into the nanosystem even after coating of gadolinium. This is an advantage of the PRIMERS with respect to drug loading.

Previous studies reported that acidic environments are generally ideal conditions to mimic tumor cell extracellular microenvironment [41]. In our study, drug release profiles were characterized in acidic environment by considering previously done investigations. In addition, to doxorubicin, other anti-cancer drug such as paclitaxel (PTX) also shows similar drug release pattern (FIG. 10), where higher release is observed in acidic environment like DOX. Previous studies demonstrate that due to protonation of the amine group of PDA in acidic environment, π-π stacking between PDA and anti-cancer drug molecules disrupt [30, 41]. In terms of therapeutic efficacy of drug loaded PDA nanobowls, a significant increase in cancer cells death was observed in previous study by in DOX loaded PDA nanbowls compared with DOX alone [30]. Similar results are observed here for PTX loaded PDA nanobowls (FIG. 11) and more importantly showing a continues release of both drugs more than 48 hours.

PRIMERS loading anti CD40 into gadolinium coated PDA nanobowl and in vitro release profile.

Polydopamine is considered as biomimetic analogues of natural photoprotective materials which has capacity to adsorb and covalently interact with proteins via supramolecular interactions including electrostatic, van der Waals or hydrogen bonds.

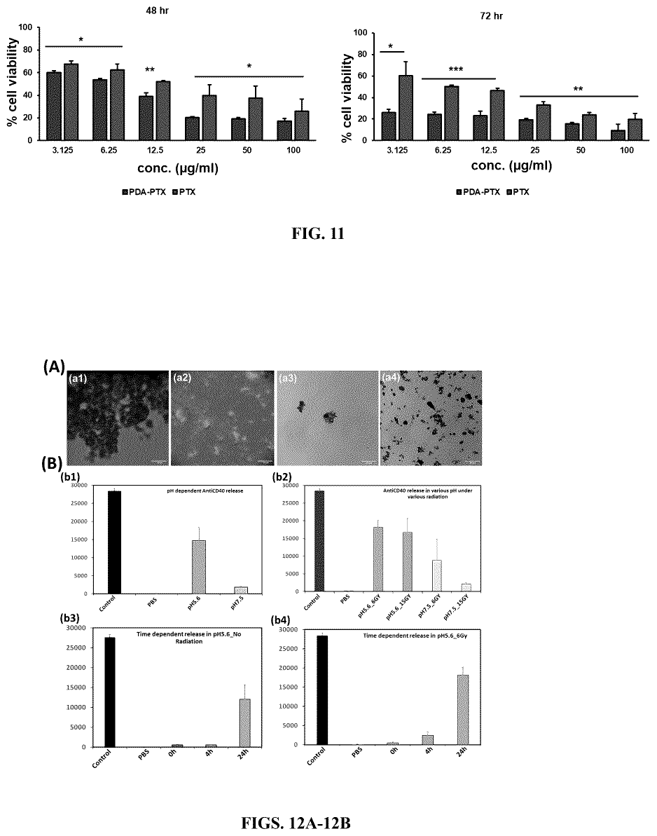

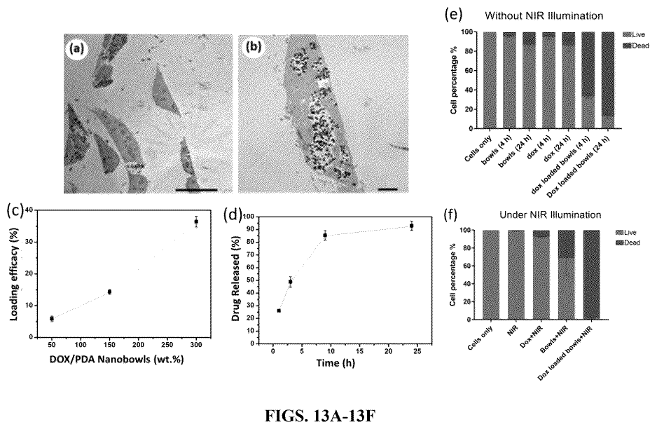

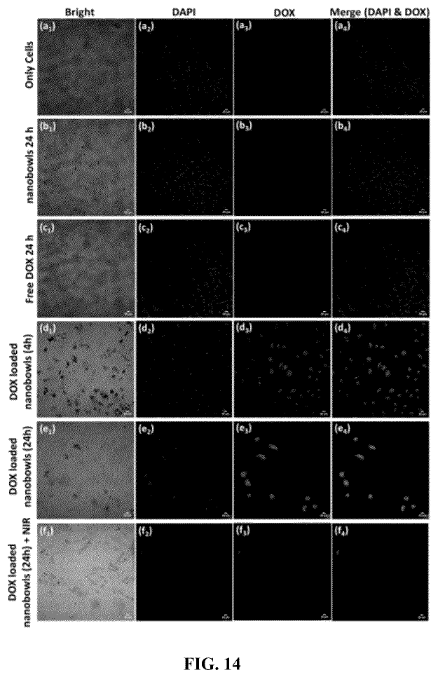

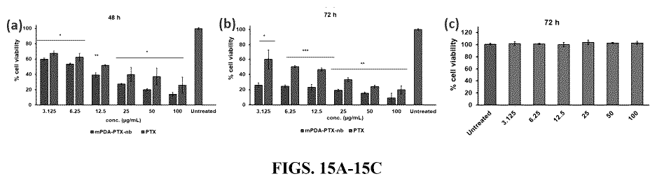

Additionally, it has been demonstrated that covalent bioconjugation to proteins could proceed when the catechol groups are oxidized to quinones that are susceptible to nucleophilic attack by amines and thiols through Michael addition and Schiff based reactions. Inspired by the protein adsorption efficiency of PDA, in our study, for the first time, we have conducted experiments in loading of AntiCD40 into the gadolinium coated PDA nanobowls. Confocal microscopy images in FIG. 12A confirm successful loading of Alexa fluor 700 Anti Human CD40 into the PDA nanobowls with gadolinium coating and non-coated ones. After several wash with MES buffer no fluorophore from Alexa fluor 700 Anti Human CD40 was observed in the supernatant confirming stability of the antiCD40 in the PDA nanobowls. Later on, in vitro release profile of antiCD40 was investigated in various environment with and without exposure to radiation (FIG. 12B). Herein, acidic environment has been found most favorable in releasing antiCD40. As observed, at pH 5.6 significant amount of AntiCD 40 is released in 24 h (FIG. 12Bb1). It is assumed that the bond between PDA and AntiCD40 disrupt in acidic environment causing release of AntiCD40. Lower pH is favorable for protonation of the amine group of PDA, which tends to disrupt the bonding between PDA and molecules [30, 41]. Remarkably, the results show higher antiCD40 release under radiation (FIG. 12B b2). Moreover, a faster release was observed under radiation compared to samples without any radiation at 4 h (FIG. 12B b3 and b4), indicating strong contribution of radiation in activating release of anti-CD40. However, there was no noticeable impact observed in various doses of radiation in this release profile. These results suggest that a certain pH environment is important in releasing AntiCD40 and radiation is beneficial in faster release in acidic environment.

### REFERENCES (DOCUMENTS CITED ABOVE BY CORRESPONDING REFERENCE NUMBER)

1. Siegel, R. L.; Miller, K. D.; Fuchs, H. E.; Jemal, A. Cancer statistics, 2022. CA Cancer J. Clin. 2022, 72, 7-33.
2. Arruebo, M.; Vilaboa, N.; Sáez-Gutierrez, B.; Lambea, J.; Tres, A.; Valladares, M.; González-Fernández, A. Assessment of the evolution of cancer treatment therapies. Cancers 2011, 3, 3279-3330.
3. Wang, X.; Zhang, H.; Chen, X. Drug resistance and combating drug resistance in cancer. Cancer Drug Resist. 2019, 2, 141-160.]
4. Pucci, C.; Martinelli, C.; Ciofani, G. Innovative approaches for cancer treatment: Current perspectives and new challenges. Ecancermedicalscience 2019, 13, 961.
5. Cheng, Z.; Li, M.; Dey, R.; Chen, Y. Nanomaterials for cancer therapy: Current progress and perspectives. J. Hematol. Oncol. 2021, 14, 85.
6. Debela, D. T.; Muzazu, S. G.; Heraro, K. D.; Ndalama, M. T.; Mesele, B. W.; Haile, D. C.; Kitui, S.K., Manyazewal, T. New approaches and procedures for cancer treatment: Current perspectives. SAGE Open Med. 2021, 9, 20503121211034366.
7. Gavas, S.; Quazi, S.; Karpi′ nski, T. M. Nanoparticles for Cancer Therapy: Current Progress and Challenges. Nanoscale Res. Lett. 2021, 16, 173.
8. Aslan, B.; Ozpolat, B.; Sood, A. K.; Lopez-Berestein, G. Nanotechnology in cancer therapy. J. Drug Target 2013, 21, 904-913.
9. Navya, P. N. ; Kaphle, A.; Srinivas, S. P.; Bhargava, S. K.; Rotello, V. M.; Daima, H. K. Current trends and challenges in cancer management and therapy using designer nanomaterials. Nano Converg. 2019, 6, 23.
10. Pillai, G.; Ceballos-Coronel, M. L. Science and technology of the emerging nanomedicines in cancer therapy: A primer for physicians and pharmacists. SAGE Open Med. 2013, 1, 2050312113513759.
11. Shukla, A.; Maiti, P. Nanomedicine and versatile therapies for cancer treatment. MedComm 2022, 3, e163.
12. Mundekkad, D.; Cho, W. C. Nanoparticles in Clinical Translation for Cancer Therapy. Int. J. Mol. Sci. 2022, 23, 1685.
13. Pandey, A. ; Nikam, A. N.; Padya, B. S.; Kulkarni, S.; Fernandes, G.; Shreya, A. B.; Garcia, M. C.; Caro, C.; Páez-Muñoz, J. M.; Dhas, N.; et al. Surface architectured black phosphorous nanoconstructs based smart and versatile platform for cancer theranostics. Coord. Chem. Rev. 2021, 435, 213826.
14. Cucinotto, I.; Fiorillo, L.; Gualtieri, S.; Arbitrio, M.; Ciliberto, D.; Staropoli, N.; Grimaldi, A.; Luce, A.; Tassone, P.; Caraglia, M.; et al. Nanoparticle albumin bound Paclitaxel in the treatment of human cancer: Nanodelivery reaches prime-time? J. Drug Deliv.
   2013, 2013, 905091.
15. Ma, P.; Mumper, R. J. Paclitaxel Nano-Delivery Systems: A Comprehensive Review. J. Nanomed. Nanotechnol. 2013, 4, 1000164.
16. Sajja, H. K.; East, M. P.; Mao, H.; Wang, Y. A.; Nie, S.; Yang, L. Development of multifunctional nanoparticles for targeted drug delivery and noninvasive imaging of therapeutic effect. Curr. Drug Discov. Technol. 2009, 6, 43-51.
17. Srinivasan, M.; Rajabi, M.; Mousa, S.A. Multifunctional Nanomaterials and Their Applications in Drug Delivery and Cancer Therapy. Nanomaterials 2015, 5, 1690-1703.
18. Caro, C.; Gámez, F.; Quaresma, P.; Páez-Muñoz, J. M.; Dominguez, A.; Pearson, J. R.; Pernia Leal, M.; Beltrán, A. M.; Fernandez-Afonso, Y.; De la Fuente, J. M.; et al. Fe3O4-Au Core-Shell Nanoparticles as a Multimodal Platform for In Vivo Imaging and Focused Photothermal Therapy. Pharmaceutics 2021, 13, 416.
19. Bertrand, N.; Wu, J.; Xu, X.; Kamaly, N.; Farokhzad, O. C. Cancer nanotechnology: The impact of passive and active targeting in the era of modern cancer biology. Adv. Drug Deliv. Rev. 2014, 66, 2-25.
20. Sriraman, S. K.; Aryasomayajula, B.; Torchilin, V. P. Barriers to drug delivery in solid tumors. Tissue Barriers 2014, 2, e29528.
21. De Jong, W. H.; Borm, P. J. Drug delivery and nanoparticles: applications and hazards. Int. J. Nanomed. 2008, 3, 133-149.
22. Matsumura, Y.; Maeda, H. A new concept for macromolecular therapeutics in cancer chemotherapy: Mechanism of tumoritropic accumulation of proteins and the antitumor agent smancs. Cancer Res. 1986, 46, 6387-6392.
23. Maeda, H.; Bharate, G. Y.; Daruwalla, J. Polymeric drugs for efficient tumor-targeted drug delivery based on EPR-effect. Eur. J. Pharm. Biopharm. 2009, 71, 409-419.
24. van der Meel, R.; Lammers, T.; Hennink, W. E. Cancer nanomedicines: Oversold or underappreciated? Expert Opin. Drug Deliv. 2017, 14, 1-5. Nanomaterials 2023, 13, 1656 16 of 19
25. Wilhelm, S.; Tavares, A.; Dai, Q.; Ohta, S.; Audet, J.; Dvorak, H.; Chan, W. Analysis of nanoparticle delivery to tumours. Nat. Rev. Mater. 2016, 1, 16014.
26. Soetaert, F.; Korangath, P.; Serantes, D.; Fiering, S.; Ivkov, R. Cancer therapy with iron oxide nanoparticles: Agents of thermal and immune therapies. Adv. Drug Deliv. Rev. 2020, 163-164, 65-83.
27. Wu, J. The Enhanced Permeability and Retention (EPR) Effect: The Significance of the Concept and Methods to Enhance Its Application. J. Pers. Med. 2021, 11, 771.
28. Nichols, J. W.; Bae, Y. H. EPR: Evidence and fallacy. J. Control. Release 2014, 190, 451-464.
29. Nakamura, Y.; Mochida, A.; Choyke, P. L.; Kobayashi, H. Nanodrug Delivery: Is the Enhanced Permeability and Retention Effect Sufficient for Curing Cancer? Bioconjug. Chem. 2016, 27, 2225-2238.
30. Korangath, P.; Barnett, J. D.; Sharma, A.; Henderson, E. T.; Stewart, J.; Yu, S. H.; Kandala, S. K.; Yang, C. T.; Caserto, J. S.; Hedayati, M.; et al. Nanoparticle interactions with immune cells dominate tumor retention and induce T cell-mediated tumor suppression in models of breast cancer. Sci. Adv. 2020, 6, eaay 1601.
31. Bao, G.; Mitragotri, S.; Tong, S. Multifunctional nanoparticles for drug delivery and molecular imaging. Annu. Rev. Biomed. Eng. 2013, 15, 253-282.
32. Yao, Y.; Zhou, Y.; Liu, L.; Xu, Y.; Chen, Q.; Wang, Y.; Wu, S.; Deng, Y.; Zhang, J.; Shao, A. Nanoparticle-Based Drug Delivery in Cancer Therapy and Its Role in Overcoming Drug Resistance. Front. Mol. Biosci. 2020, 7, 193.
33. Xin, Y.; Yin, M.; Zhao, L.; Meng, F.; Luo, L. Recent progress on nanoparticle-based drug delivery systems for cancer therapy. Cancer Biol. Med. 2017, 14, 228-241.
34. Ngwa, W.; Kumar, R.; Moreau, M.; Dabney, R.; Herman, A. Nanoparticle Drones to Target Lung Cancer with Radiosensitizers and Cannabinoids. Front. Oncol. 2017, 7, 208.
35. Ridolfo, R.; Tavakoli, S.; Junnuthula, V.; Williams, D. S.; Urtti, A.; van Hest, J. C. M. Exploring the Impact of Morphology on the Properties of Biodegradable Nanoparticles and Their Diffusion in Complex Biological Medium. Biomacromolecules 2021, 22, 126-133.
36. Caldorera-Moore, M.; Guimard, N.; Shi, L.; Roy, K. Designer nanoparticles: Incorporating size, shape and triggered release into nanoscale drug carriers. Expert Opin. Drug Deliv. 2010, 7, 479-495.
37. Murugan, K.; Choonara, Y. E.; Kumar, P.; Bijukumar, D.; du Toit, L. C.; Pillay, V. Parameters and characteristics governing cellular internalization and trans-barrier trafficking of nanostructures. Int. J. Nanomed. 2015, 10, 2191-2206.
38. Kohout, C.; Santi, C.; Polito, L. Anisotropic Gold Nanoparticles in Biomedical Applications. Int. J. Mol. Sci. 2018, 19, 3385.
39. Jindal, A. The effect of particle shape on cellular interaction and drug delivery applications of micro-and nanoparticles. Int. J. Pharm. 2017, 532, 450-465.
40. Burrows, N. D.; Vartanian, A. M.; Abadeer, N. S.; Grzincic, E. M.; Jacob, L. M.; Lin, W.; Li, J.; Dennison, J. M.; Hinman, J. G.; Murphy, C. J. Anisotropic Nanoparticles and Anisotropic Surface Chemistry. J. Phys. Chem. Lett. 2016, 7, 632-641.
41. Acter, S.; Cho, J.; Kim, J.; Byun, A.; Park, K. H. Synthesis and Shape Control of Uniform Polymer Microparticles by Tailored Adsorption of Poly(ethylene oxide)-b-Poly(“-caprolactone) Copolymer. Bull. Korean Chem. Soc. 2015, 36, 1467-1473.
42. Zhang, L.; Su, H.; Wang, H.; Li, Q.; Li, X.; Zhou, C.; Xu, J.; Chai, Y.; Liang, X.; Xiong, L.; et al. Tumor Chemo-Radiotherapy with Rod-Shaped and Spherical Gold Nano Probes: Shape and Active Targeting Both Matter. Theranostics 2019, 9, 1893-1908.
43. Guan, B. Y.; Zhang, S. L.; Lou, X. W. Realization ofWalnut-Shaped Particles with Macro-/Mesoporous Open Channels through Pore Architecture Manipulation and Their Use in Electrocatalytic Oxygen Reduction. Angew. Chem. Int. Ed. 2018, 57, 6176-6180.
44. Guan, B. Y.; Yu, L.; Lou, X. W. Formation of Asymmetric Bowl-Like Mesoporous Particles via Emulsion-Induced Interface Anisotropic Assembly. J. Am. Chem. Soc. 2016, 138, 11306-11311.
45. Li, H.; Zhang, W.; Tong, W.; Gao, C. Enhanced Cellular Uptake of Bowl-like Microcapsules. ACS Appl. Mater. Interfaces 2016, 8, 11210-11214.
46. Acter, S.; Vidallon, M. L. P.; Crawford, S.; Tabor, R. F.; Teo, B. M. Efficient Cellular Internalization and Transport of Bowl-Shaped Polydopamine Particles. Part. Part. Syst. Charact. 2020, 37, 2000166.
47. Xie, X.; Liao, J.; Shao, X.; Li, Q.; Lin, Y. The Effect of shape on Cellular Uptake of Gold Nanoparticles in the forms of Stars, Rods, and Triangles. Sci. Rep. 2017, 7, 3827.
48. Nangia, S.; Sureshkumar, R. Effects of Nanoparticle Charge and Shape Anisotropy on Translocation through Cell Membranes. Langmuir 2012, 28, 17666-17671.
49. Meyer, R. A.; Green, J. J. Shaping the future of nanomedicine: Anisotropy in polymeric nanoparticle design. WIREs Nanomed. Nanobiotechnol. 2016, 8, 191-207.
50. Lu, Z., Acter, S.; Teo, B. M.; Tabor, R. F. Synthesis and characterisation of polynorepinephrine-shelled microcapsules via an oil-in-water emulsion templating route. J. Mater. Chem. B 2021, 9, 9575-9582.
51. Lungu, I. I.; Grumezescu, A. M.; Volceanov, A.; Andronescu, E. Nanobiomaterials Used in Cancer Therapy: An Up-to-Date Overview. Molecules 2019, 24, 3547.
52. Ball, V. Polydopamine Nanomaterials: Recent Advances in Synthesis Methods and Applications. Front. Bioeng. Biotechnol. 2018, 6, 109. Nanomaterials 2023, 13, 1656 17 of 19
53. Jin, A.; Wang, Y.; Lin, K.; Jiang, L. Nanoparticles modified by polydopamine: Working as “drug” carriers. Bioact. Mater. 2020, 5, 522-541.
54. Lu, Z.; Acter, S.; Teo, B. M.; Bishop, A. I.; Tabor, R. F.; Vidallon, M. L. P. Mesoporous, anisotropic nanostructures from bioinspired polymeric catecholamine neurotransmitters and their potential application as photoacoustic imaging agents. J. Mater. Chem. B 2022, 10, 9662-9670.
55. Liu, Y.; Ai, K.; Lu, L. Polydopamine and Its Derivative Materials: Synthesis and Promising Applications in Energy, Environmental, and Biomedical Fields. Chem. Rev. 2014, 114, 5057-5115.
56. Garcia-Pardo, J.; Novio, F.; Nador, F.; Cavaliere, I.; Suárez-Garcia, S.; Lope-Piedrafita, S.; Candiota, A. P.; Romero-Gimenez, J.; Rodriguez-Galván, B.; Bové, J.; et al. Bioinspired Theranostic Coordination Polymer Nanoparticles for Intranasal Dopamine Replacement in Parkinson's Disease. ACS Nano 2021, 15, 8592-8609.
57. Monge-Fuentes, V.; Biolchi Mayer, A.; Lima, M. R.; Geraldes, L. R.; Zanotto, L. N.; Moreira, K. G.; Martins, O. P.; Piva, H. L.; Felipe, M. S. S.; Amaral, A. C.; et al. Dopamine-loaded nanoparticle systems circumvent the blood-brain barrier restoring motor function in mouse model for Parkinson's Disease. Sci. Rep. 2021, 11, 15185.
58. Cheng, W.; Zeng, X.; Chen, H.; Li, Z.; Zeng, W.; Mei, L.; Zhao, Y. Versatile Polydopamine Platforms: Synthesis and Promising Applications for Surface Modification and Advanced Nanomedicine. ACS Nano 2019, 13, 8537-8565.
59. Ochs, C. J.; Hong, T.; Such, G. K.; Cui, J.; Postma, A.; Caruso, F. Dopamine-Mediated Continuous Assembly of Biodegradable Capsules. Chem. Mater. 2011, 23, 3141-3143.
60. Mousavi, S. M.; Zarei, M.; Hashemi, S. A. Polydopamine for Biomedical Application and Drug Delivery System. Med. Chem. 2018, 8, 218-229.
61. Zhu, Z.; Su, M. Polydopamine Nanoparticles for Combined Chemo-and Photothermal Cancer Therapy. Nanomaterials 2017, 7, 160.
62. Acter, S.; Jahan, N.; Vidallon, M. L. P.; Teo, B. M.; Tabor, R. F. Mesoporous Polydopamine Nanobowls Toward Combined Chemoand Photothermal Cancer Therapy. Part. Part. Syst. Charact. 2022, 39, 2200015.
63. Acter, S.; Vidallon, M. L. P.; King, J. P.; Teo, B. M.; Tabor, R. F. Photothermally responsive Pickering emulsions stabilised by polydopamine nanobowls. J. Mater. Chem. B 2021, 9, 8962-8970.
64. Siciliano, G.; Monteduro, A. G.; Turco, A.; Primiceri, E.; Rizzato, S.; Depalo, N.; Curri, M. L.; Maruccio, G. Polydopamine-Coated Magnetic Iron Oxide Nanoparticles: From Design to Applications. Nanomaterials 2022, 12, 1145.
65. Ahuja, U.; Wang, B.; Hu, P.; Rethore, J.; Aifantis, K. E. Polydopamine coated Si nanoparticles allow for improved mechanical and electrochemical stability. Electrochim. Acta 2021, 392, 138993.
66. Mrówczy′ nski, R. Polydopamine-Based Multifunctional (Nano)materials for Cancer Therapy. ACS Appl. Mater. Interfaces 2018, 10, 7541-7561.
67. Black, K.; Yi, J.; Rivera, J.; Zelasko-Leon, D.; Messersmith, P. Polydopamine-enabled surface functionalization of gold nanorods for cancer cell targeting and thermal ablation. Nanomedicine 2012, 8, 17-28.
68. Niu, G.; Zhao, L.; Wang, Y.; Jiang, Y. PDA/gold nanorod-based nanoparticles for synergistic genetic and photothermal combination therapy for cancer treatment. ChemPhysMater 2023, 2, 83-89.
69. You, Y. H.; Lin, Y. F.; Nirosha, B.; Chang, H. T.; Huang, Y. F. Polydopamine-coated gold nanostar for combined antitumor and antiangiogenic therapy in multidrug-resistant breast cancer. Nanotheranostics 2019, 3, 266-283.
70. Khlebtsov, B. N.; Burov, A. M.; Pylaev, T. E.; Khlebtsov, N. G. Polydopamine-coated Au nanorods for targeted fluorescent cell imaging and photothermal therapy. Beilstein J. Nanotechnol. 2019, 10, 794-803.
71. Cheng, Y.; Zhang, S.; Kang, N.; Huang, J.; Lv, X.; Wen, K.; Ye, S.; Chen, Z.; Zhou, X.; Ren, L. Polydopamine-Coated Manganese Carbonate Nanoparticles for Amplified Magnetic Resonance Imaging-Guided Photothermal Therapy. ACS Appl. Mater. Interfaces 2017, 9, 19296-19306.
72. Lin, L.-S.; Cong, Z.-X.; Cao, J.-B.; Ke, K.-M.; Peng, Q.-L.; Gao, J.; Yang, H.-H.; Liu, G.; Chen, X. Multifunctional Fe3O4@Polydopamine Core-Shell Nanocomposites for Intracellular mRNA Detection and Imaging-Guided Photothermal Therapy. ACS Nano 2014, 8, 3876-3883.
73. Korchinski, D. J.; Taha, M.; Yang, R., Nathoo, N.; Dunn, J. F. Iron Oxide as an MRI Contrast Agent for Cell Tracking. Magn. Reason. Insights 2015, 8, 15-29.
74. Perlman, O.; Borodetsky, A.; Kauffmann, Y.; Shamay, Y.; Azhari, H.; Weitz, I. S. Gold/Copper@Polydopamine Nanocomposite for Contrast-Enhanced Dual Modal Computed Tomography-Magnetic Resonance Imaging. ACS Appl. Nano Mater. 2019, 2, 6124-6134.
75. Luo, D.; Wang, X.; Burda, C.; Basilion, J. P. Recent Development of Gold Nanoparticles as Contrast Agents for Cancer Diagnosis. Cancers 2021, 13, 1825.
76. Wang, W.; Tang, Z.; Zhang, Y.; Wang, Q.; Liang, Z.; Zeng, X. Mussel-Inspired Polydopamine: The Bridge for Targeting Drug Delivery System and Synergistic Cancer Treatment. Macromol. Biosci. 2020, 20, 2000222.
77. Carmignani, A.; Battaglini, M.; Sinibaldi, E.; Marino, A.; Vighetto, V.; Valentina, C.; Ciofani, G. In Vitro and Ex Vivo Investigation of the Effects of Polydopamine Nanoparticle Size on Their Antioxidant and Photothermal Properties: Implications for Biomedical Applications. ACS Appl. Nano Mater. 2022, 5, 1702-1713.
78. Liebscher, J.; Mrówczy′ nski, R.; Scheidt, H. A.; Filip, C.; Hadade, N. D.; Turcu, R.; Bende, A.; Beck, S. Structure of Polydopamine: A Never-Ending Story? Langmuir 2013, 29, 10539-10548. Nanomaterials 2023, 13, 1656 18 of 19
79. Acter, S.; Vidallon, M. L. P.; Crawford, S.; Tabor, R. F.; Teo, B. M. Bowl-Shaped Mesoporous Polydopamine Nanoparticles for Size-Dependent Endocytosis into Hela Cells. ACS Appl. Nano Mater. 2021, 4, 9536-9546.
80. Xing, Y.; Zhang, J.; Chen, F.; Liu, J.; Cai, K. Mesoporous polydopamine nanoparticles with co-delivery function for overcoming multidrug resistance via synergistic chemo-photothermal therapy. Nanoscale 2017, 9, 8781-8790.
81. Sun, L.; Li, Q.; Zhang, L.; Xu, Z.; Kang, Y.; Xue, P. PEGylated Polydopamine Nanoparticles Incorporated with Indocyanine Green and Doxorubicin for Magnetically Guided Multimodal Cancer Therapy Triggered by Near-Infrared Light. ACS Appl. Nano Mater. 2018, 1, 325-336.
82. Zhao, Y.; Yeh, Y.; Liu, R.; You, J.; Qu, F. Facile deposition of gold nanoparticles on core-shell Fe3O4@polydopamine as recyclable nanocatalyst. Solid State Sci. 2015, 45, 9-14.
83. Zheng, X.; Zhang, J.; Wang, J.; Qi, X.; Rosenholm, J. M.; Cai, K. Polydopamine Coatings in Confined Nanopore Space: Toward Improved Retention and Release of Hydrophilic Cargo. J. Phys. Chem. C 2015, 119, 24512-24521.
84. Busa, P.; Koutavarapu, R., Kuthati, Y. Polydopamine-Coated Copper-Substituted Mesoporous Silica Nanoparticles for Dual Cancer Therapy. Coatings 2022, 12, 60.
85. Dong, Z.; Gong, H.; Gao, M.; Zhu, W.; Sun, X.; Feng, L.; Fu, T.; Li, Y.; Liu, Z. Polydopamine Nanoparticles as a Versatile Molecular Loading Platform to Enable Imaging-guided Cancer Combination Therapy. Theranostics 2016, 6, 1031-1042.
86. Zhuang, H.; Su, H.; Bi, X.; Bai, Y.; Chen, L.; Ge, D.; Shi, W.; Sun, Y. Polydopamine Nanocapsule: A Theranostic Agent for Photoacoustic Imaging and Chemo-Photothermal Synergistic Therapy. ACS Biomater. Sci. Eng. 2017, 3, 1799-1808.
87. Chen, X.; Yan, Y.; Müllner, M.; van Koeverden, M. P.; Noi, K. F.; Zhu, W.; Caruso, F. Engineering Fluorescent Poly(dopamine) Capsules. Langmuir 2014, 30, 2921-2925.
88. Wood, B. R.; Langford, S. J.; Cooke, B. M.; Lim, J.; Glenister, F. K.; Duriska, M.; Unthank, J. K.; McNaughton, D. Resonance Raman Spectroscopy Reveals New Insight into the Electronic Structure of _-Hematin and Malaria Pigment. J. Am. Chem. Soc. 2004, 126, 9233-9239.
89. Yuen, C.; Liu, Q. Magnetic field enriched surface enhanced resonance Raman spectroscopy for early malaria diagnosis. J. Biomed. Opt. 2012, 17, 017005.
90. Liu, X.; Cao, J.; Li, H.; Li, J.; Jin, Q.; Ren, K.; Ji, J. Mussel-Inspired Polydopamine: A Biocompatible and Ultrastable Coating for Nanoparticles in Vivo. ACS Nano 2013, 7, 9384-9395.
91. Zhou, X.; Chang, T. L.; Chen, S.; Liu, T.; Wang, H.; Liang, J. F. Polydopamine-Decorated Orlistat-Loaded Hollow Capsules with an Enhanced Cytotoxicity against Cancer Cell Lines. Mol. Pharm. 2019, 16, 2511-2521.
92. Lu, Z.; Douek, A. M.; Rozario, A. M.; Tabor, R. F.; Kaslin, J.; Follink, B.; Teo, B. M. Bioinspired polynorepinephrine nanoparticles as an efficient vehicle for enhanced drug delivery. J. Mater. Chem. B 2020, 8, 961-968.
93. Liu, F.; He, X.; Zhang, J.; Chen, H.; Zhang, H.; Wang, Z. Controllable synthesis of polydopamine nanoparticles in microemulsions with pH-activatable properties for cancer detection and treatment. J. Mater. Chem. B 2015, 3, 6731-6739.
94. Di, J.; Gao, X.; Du, Y.; Zhang, H.; Gao, J.; Zheng, A. Size, shape, charge and “stealthy” surface: Carrier properties affect the drug circulation time in vivo. Asian J. Pharm. Sci. 2021, 16, 444-458.
95. Foroozandeh, P.; Aziz, A.A. Insight into Cellular Uptake and Intracellular Trafficking of Nanoparticles. Nanoscale Res. Lett. 2018, 13, 339.
96. Chen, F.; Xing, Y.; Wang, Z.; Zheng, X.; Zhang, J.; Cai, K. Nanoscale Polydopamine (PDA) Meets _-_ Interactions: An Interface-Directed Coassembly Approach for Mesoporous Nanoparticles. Langmuir 2016, 32, 12119-12128.
97. Ryu, J. H.; Messersmith, P. B.; Lee, H. Polydopamine Surface Chemistry: A Decade of Discovery. ACS Appl. Mater. Interfaces 2018, 10, 7523-7540.
98. Pada, A. K.; Desai, D.; Sun, K.; Prakirth Govardhanam, N.; Törnquist, K.; Zhang, J.; Rosenholm, J. M. Comparison of Polydopamine-Coated Mesoporous Silica Nanorods and Spheres for the Delivery of Hydrophilic and Hydrophobic Anticancer Drugs. Int. J. Mol. Sci. 2019, 20, 3408.
99. Li, J.; Zhang, Z.; Deng, H.; Zheng, Z. Cinobufagin-Loaded and Folic Acid-Modified Polydopamine Nanomedicine Combined With Photothermal Therapy for the Treatment of Lung Cancer. Front. Chem. 2021, 9, 637754.
100. Hu, H.; Liu, X.; Hong, J.; Ye, N.; Xiao, C.; Wang, J.; Li, Z.; Xu, D. Mesoporous polydopamine-based multifunctional nanoparticles for enhanced cancer phototherapy. J. Colloid Interface Sci. 2022, 612, 246-260.
101. Zhang, J.; Zhang, K.; Hao, Y.; Yang, H.; Wang, J.; Zhang, Y.; Zhao, W.; Ma, S.; Mao, C. Polydopamine nanomotors loaded indocyanine green and ferric ion for photothermal and photodynamic synergistic therapy of tumor. J. Colloid Interface Sci. 2023, 633, 679-690.
102. Li, S.; Chen, Y.; He, P.; Ma, Y.; Cai, Y.; Hou, X.; Zhang, G., Zhang, X.; Wang, Z. Aggregation-Induced Emission (AIE) Photosensitizer Combined Polydopamine Nanomaterials for Organelle-Targeting Photodynamic and Photothermal Therapy by the Recognition of Sialic Acid. Adv. Healthc. Mater. 2022, 11, 2200242.
103. Ngwa, W.; Irabor, O. C.; Schoenfeld, J. D.; Hesser, J.; Demaria, S.; Formenti, S. C. Using immunotherapy to boost the abscopal effect. Nat. Rev. Cancer 2018, 18, 313-322.
104. Hao, Y.; Altundal, Y.; Moreau, M.; Sajo, E.; Kumar, R.; Ngwa, W. Potential for enhancing external beam radiotherapy for lung cancer using high-Z nanoparticles administered via inhalation. Phys. Med. Biol. 2015, 60, 7035-7043. Nanomaterials 2023, 13, 1656 19 of 19
105. Hao, Y.; Yasmin-Karim, S.; Moreau, M.; Sinha, N.; Sajo, E.; Ngwa, W. Enhancing radiotherapy for lung cancer using immunoadjuvants delivered in situ from new design radiotherapy biomaterials: A preclinical study. Phys. Med. Biol. 2016, 61, N697-N707.
106. Yasmin-Karim, S.; Bruck, P. T.; Moreau, M.; Kunjachan, S.; Chen, G. Z.; Kumar, R.; Grabow, S.; Dougan, S. K.; Ngwa, W. Radiation and Local Anti-CD40 Generate an Effective in situ Vaccine in Preclinical Models of Pancreatic Cancer. Front. Immunol. 2018, 9, 2030.
107. Wang, Z.; Wang, Y.; Wang, Y.; Wei, C.; Deng, Y.; Chen, H.; Shen, J.; Ke, H. Biomineralized iron oxide-polydopamine hybrid nanodots for contrast-enhanced T1-weighted magnetic resonance imaging and photothermal tumor ablation. J. Mater. Chem. B 2021, 9, 1781-1786.
108. Nkanga, C. I.; Ortega-Rivera, O. A.; Steinmetz, N. F. Photothermal immunotherapy of melanoma using TLR-7 agonist laden tobacco mosaic virus with polydopamine coat. Nanomedicine 2022, 44, 102573.
109. Wang, N.; Yang, Y.; Wang, X.; Tian, X.; Qin, W.; Wang, X.; Liang, J.; Zhang, H.; Leng, X. Polydopamine as the Antigen Delivery Nanocarrier for Enhanced Immune Response in Tumor Immunotherapy. ACS Biomater. Sci. Eng. 2019, 5, 2330-2342.
110. Zhu, M.; Shi, Y.; Shan, Y.; Guo, J.; Song, X.; Wu, Y.; Wu, M.; Lu, Y.; Chen, W.; Xu, X.; et al. Recent developments in mesoporous polydopamine-derived nanoplatforms for cancer theranostics. J. Nanobiotechnol. 2021, 19, 387.
111. Wang, S.; Lin, Q.; Chen, J.; Gao, H.; Fu, D.; Shen, S. Biocompatible polydopamine-encapsulated gadolinium-loaded carbon nanotubes for MRI and color mapping guided photothermal dissection of tumor metastasis. Carbon 2017, 112, 53-62.
112. Mueller, R.; Moreau, M.; Yasmin-Karim, S.; Protti, A.; Tillement, O.; Berbeco, R.; Hesser, J.; Ngwa, W. Imaging and Characterization of Sustained Gadolinium Nanoparticle Release from Next Generation Radiotherapy Biomaterial. Nanomaterials 2020, 10, 2249.
113. Mao, W.; Hu, C.; Zheng, H.; Xie, J.; Shi, X.; Du, Y.; Wang, F. A Functionalized Polydopamine Theranostic Nanoprobe for Efficient Imaging of miRNA-21 and In Vivo Synergetic Cancer Therapy. Mol. Ther.-Nucleic Acids 2020, 22, 27-37.
114. Chen, R., Zhu, C.; Fan, Y.; Feng, W.; Wang, J.; Shang, E.; Zhou, Q.; Chen, Z. Polydopamine-Based Multifunctional Platform for Combined Photothermal Therapy, Chemotherapy, and Immunotherapy in Malignant Tumor Treatment. ACS Appl. Bio Mater. 2019, 2, 874-883.
115. Dorsey, J. F.; Sun, L.; Joh, D. Y.; Witztum, A.; Kao, G.D.; Alonso-Basanta, M.; Avery, S.; Hahn, S. M.; Al Zaki, A.; Tsourkas, A. Gold nanoparticles in radiation research: Potential applications for imaging and radiosensitization. Transl. Cancer Res. 2013, 2, 280-291.
116. Yasmin-Karim, S.; Wood, J.; Wirtz, J.; Moreau, M.; Bih, N.; Swanson, W.; Muflam, A.; Ainsworth, V.; Ziberi, B.; Ngwa, W. Optimizing In Situ Vaccination During Radiotherapy. Front. Oncol. 2021, 11, 711078.
117. Wood, J.; Yasmin-Karim, S.; Mueller, R.; Viswanathan, A. N.; Ngwa, W. Single Radiotherapy Fraction with Local Anti-CD40 Therapy Generates Effective Abscopal Responses in Mouse Models of Cervical Cancer. Cancers 2020, 12, 1026.

### Other Embodiments

From the foregoing description, it will be apparent that variations and modifications may be made to the disclosure described herein to adopt it to various usages and conditions. Such embodiments are also within the scope of the following claims.

All citations to sequences, patents and publications in this specification are herein incorporated by reference to the same extent as if each independent patent and publication was specifically and individually indicated to be incorporated by reference. By their citation of various references in this document, Applicants do not admit any particular reference is “prior art” to their disclosure.

What is claimed:

1. A treatment method comprising,
administering to a subject in need thereof a polydopamine composition comprising one or more therapeutic agents; and thereafter
administering radiation to the patient.

2. A treatment method comprising:
a) administering a polydopamine composition comprising one or more therapeutic agents to a subject in need thereof;
b) acquiring images of a treatment site; and
c) administering radiation to the subject.

3. The method of claim 1 or 2 wherein the administered radiation is external radiotherapy.

4. The method of any one of claims 1 to 3 wherein external-beam radiotherapy (EBRT) is administered to the subject.

5. The method of any one of claims 1 to 4 wherein the radiation is administered based on images acquired of the treatment site.

6. The method of any one of claims 1 to 5 wherein treatment of the subject is monitored and/or adjusted based on imaging of the subject.

7. The method of any one of claims 1 to 6 wherein the radiation source is adjusted and/or aligned based on images of a tumor of the subject.

8. The method of any one of claims 1 to 7 wherein Cerenkov radiation is applied to the subject.

9. The method of any one of claims 1 to 8 wherein the subject is monitored based on imaging of the subject.

10. The method of any one of claims 1 to 9 wherein the polydopamine material comprises polydopamine nanoparticles.

11. The method of any one of claims 1 to 10 wherein the polydopamine material comprises polydopamine nanobowl particles.

12. The method of any one of claims 1 to 11 wherein the polydopamine material comprises an imaging-tracking agent.

13. The method of any one of claims 1 to 12 wherein the polydopamine material comprises a metal to facilitate imaging of the polydopamine material.

14. The method of any one of claims 1 to 13 wherein the polydopamine material comprises gadolinium.

15. The method of any one of claims 1 to 14 wherein the images of a treatment site are acquired by magnetic resonance imaging.

16. The method of any one of claims 1 to 15 wherein the polydopamine material is covalently bonded to one or more therapeutic agents.

17. The method of any one of claims 1 to 16 wherein the polydopamine material is non-covalently bonded to one or more therapeutic agents.

18. The method of claim 17 wherein the one or more therapeutic agents are hydrogen bonded to the polydopamine material.

19. The method of any one of claims 1 to 18 wherein the polydopamine material comprises one or more an immune adjuvant drugs.

20. The method of any one of claims 1 to 19 with the polydopamine material comprises one or more STING agonists or anti-CD40.

21. The method of any one of claims 1 to 20 with the polydopamine material comprises one or more checkpoint inhibitors.

22. The method of any one of claims 1 to 21 wherein the polydopamine material comprises Pembrolizumab, Nivolumab, Atezolizumab, Ipilimumab, Tremelimumab, Avelumab, Durvalumab, Cemiplimab and/or Dostarlimab.

23. The method of any one of claims 1 to 22 with the polydopamine comprises one or more of docetaxel, paclitaxel, doxorubicin, cisplatin, gemcitabine, a hydrophobic drug, an anti-androgen compound, a small molecule signaling pathway inhibitor, and combinations thereof.

24. The method of any one of claims 1 to 23 with the polydopamine comprises one or more of enzalutamide, flutamide, nilutamide, bicalutamide, abiraterone (including abiraterone acetate), cyproterone (including cyproterone acetate), megestrol (including megestrol acetate), chlormadinone (including chlormadinone acetate), spironolactone, canrenone, drospirenone, dienogest, norgestimate, ketoconazole, cimetidine, and combinations thereof.

25. The method of any of claims 1 to 24, wherein the subject is imaged by one or moe photoacoustic imaging, surface enhanced Raman spectroscopy, X-ray computed tomography, magnetic resonance imaging, positron emission tomography, single-photon emission computed tomography, fluorescence imaging, optical coherence tomography, or ultrasound imaging.

26. The method of any one of claims 1 to 25 wherein the administered radiation is internal radiotherapy

27. The method of any one of claims 1 to 26 wherein brachytherapy is performed.

28. The method of any one of claims 1 to 27 wherein the polydopamine material comprises a metal.

29. The method of any one of claims 1 to 28 wherein the polydopamine material comprises gadolinium.

30. The method of any one of claims 1 to 29 wherein the polydopamine material comprises gold.

31. The method of any one of claims 1 to 30 wherein the dose of radiation is less than would be administered for radiation therapy alone.

32. The method of any one of claims 1 to 31 wherein the subject has cancer.

33. The method of any one of claims 1 to 32 wherein the subject has been identified as suffering from cancer.

34. The method of claim 32 or 33 wherein the cancer is prostate cancer, lung cancer, pancreatic cancer, liver cancer, breast cancer, ovarian cancer, gastric cancer, colon cancer, brain cancer, or head and neck cancer.

35. The method of any one of claims 32 to 34 wherein the cancer is metastatic cancer.

36. A system for use in a method of any one of claims 1 to 35.

37. A polydopamine material comprising one or more STING agonists or anti-CD40.

38. The polydopamine material of claim 37 further comprising an imaging contrast agent.

39. The polydopamine material of claim 38 wherein the contrast agent is a magnetic resonance imaging (MRI) contrast agent.

40. The polydopamine material of any one of claims 37 to 39 wherein the polydopamine material comprises gadolinium.

41. The polydopamine material of an one of claims 37 to 40 wherein the polydopamine material comprises gold.

42. The polydopamine material of any one of claims 37 to 41 wherein the polydopamine material comprises polydopamine nanobowl particles

43. The polydopamine material of any one of claims 37 to 42 wherein the polydopamine material comprises one or more STING agonists.

44. The polydopamine material of any one of claims 37 to 43 wherein the polydopamine material comprises an anti-CD40 agent.

45. A polydopamine material comprising one or more checkpoint inhibitors.

46. The polydopamine material of claim 45 further comprising an imaging contrast agent.

47. The polydopamine material of claim 46 herein the contrast agent is a magnetic resonance imaging (MRI) contrast agent.

48. The polydopamine material of any one of claims 45 to 47 wherein the polydopamine material comprises gadolinium.

49. The polydopamine material of any one of claims 45 to 48 wherein the polydopamine material comprises gold.

50. The polydopamine material of any one of claims 45 to 49 wherein the polydopamine material comprises polydopamine nanobowl particles

51. The polydopamine material of any one of claims 45 to 50 wherein the polydopamine material comprises one or more STING agonists.

52. The polydopamine material of any one of claims 45 to 51 wherein the polydopamine material comprises an anti-CD40 agent.

53. The polydopamine material of any one of claims 45 to 52 wherein the polydopamine material comprises Pembrolizumab, Nivolumab, Atezolizumab, Ipilimumab, Tremelimumab, Avelumab, Durvalumab, Cemiplimab and/or Dostarlimab.

54. A polydopamine material comprising one or more of docetaxel, paclitaxel, doxorubicin, cisplatin, gemcitabine, enzalutamide, flutamide, nilutamide, bicalutamide, abiraterone (including abiraterone acetate), cyproterone (including cyproterone acetate), megestrol (including megestrol acetate), chlormadinone (including chlormadinone acetate), spironolactone, canrenone, drospirenone, dienogest, norgestimate, ketoconazole, r cimetidine, and combinations thereof.

55. The polydopamine material of claim 54 further comprising an imaging contrast agent.

56. The polydopamine material of claim 55 wherein the contrast agent is a magnetic resonance imaging (MRI) contrast agent.

57. The polydopamine material of any one of claims 54 to 56 wherein the polydopamine material comprises gadolinium.

58. The polydopamine material of any one of claims 54 to 57 wherein the polydopamine material comprises gold.

59. The polydopamine material of any one of claims 54 to 58 wherein the polydopamine material comprises polydopamine nanobowl particles

60. The polydopamine material of any one of claims 54 to 59 wherein the polydopamine material comprises one or more STING agonists.

61. The polydopamine material of any one of claims 54 to 60 wherein the polydopamine material comprises an anti-CD40 agent.

62. The polydopamine material of any one of claims 54 to 61 wherein the polydopamine material comprises Pembrolizumab, Nivolumab, Atezolizumab, Ipilimumab, Tremelimumab, Avelumab, Durvalumab, Cemiplimab and/or Dostarlimab.
